(19) United States
(12) Patent Application Publication
(10) Pub. No.: US 2004/0071656 A1
(43) Pub. Date: Apr. 15, 2004

(54) Modulation of heat-shock-protein-based immunotherapies

(76) Inventors: Felix Wieland, Heidelberg (DE); Franz-Ulrich Hartl, Kottgeisering (DE)

Correspondence Address: KENYON & KENYON, ONE BROADWAY, NEW YORK, NY, 10004, US

(21) Appl. No.: 10/328,953

(22) Filed: Dec. 23, 2002

Related U.S. Application Data
(60) Provisional application No. 60/342,570, filed on Dec. 26, 2001.
(60) Provisional application No. 60/343,884, filed on Dec. 27, 2001.
(60) Provisional application No. 60/372,620, filed on Apr. 12, 2002.
(60) Provisional application No. 60/399,342, filed on Jul. 29, 2002.
(60) Provisional application No. 60/414,834, filed on Sep. 28, 2002.

(51) Int. Cl. A61K 39/00; A61K 38/19; A61K 38/20

(52) U.S. Cl. 424/85.1; 424/85.2; 424/185.1

(57) ABSTRACT

Methods and compositions are provided for modulating the immune response to an antigen based upon the finding that the cell surface protein CD40 is a mammalian heat shock protein (hsp) receptor. Cell surface CD40 mediates the binding, cell signaling, and uptake of hsp and particularly hsp with antigen bound thereto. Methods are provided for modulating hsp-antigen uptake and an immune response to the antigen by altering CD40 expression, as well as utilizing CD40-binding fragments of mammalian hsp and muteins thereof for targeting antigens to CD40-expressing cells. Screening methods for agonists and antagonists of the CD40-hsp interaction are also provided.

### CROSS-REFERENCE TO RELATED APPLICATIONS

Priority is claimed under 35 USC § 119 and § 120 to prior Provisional Applications No. 60/342,570, filed Dec. 26, 2001; 60/343,884, filed Dec. 28, 2001; 60/372,620, filed Apr. 12, 2002, 60/399,342, filed Jul. 29, 2002, and 60/414,834, filed Sep. 28, 2002, all of which are incorporated herein by reference in their entireties.

### BACKGROUND OF THE INVENTION

Heat shock proteins (“hsps”) constitute a highly conserved class of proteins selectively induced in cells under stressful conditions, such as sudden increases in temperature or glucose deprivation. Able to bind to a wide variety of other proteins in their non-native states, heat shock proteins participate in the genesis of these proteins, including their synthesis, folding, assembly, disassembly and translocation (Freeman and Morimoto, 1996, EMBO J. 15:2969-2979; Lindquist and Craig, 1988, Annu. Rev. Genet. 22:631-677; Hendrick and Hartl, 1993, Annu. Rev. Biochem. 62:349-384). Because they guide other proteins through the biosynthetic pathway, heat shock proteins are said to function as “molecular chaperones” (Frydman et al., 1994, Nature 370:111-117; Hendrick and Hartl, Annu. Rev. Biochem. 62:349-384; Hartl, 1996, Nature 381:571-580). Induction during stress is consistent With their chaperone function; for example, dnaK, the Escherichia coli hsp70 homolog, is able to reactivate heat-inactivated RNA polymerase (Ziemienowicz et al., 1993, J. Biol. Chem. 268:25425-25341).

It has been proposed that gp96 may assist in the assembly of multi-subunit proteins in the endoplasmic reticulum (Wiech et al., 1992, Nature 358:169-170). Indeed, gp96 has been observed to associate with unassembled immunoglobulin chains, major histocompatability class II molecules, and a mutant glycoprotein B from Herpes simplex virus (Melnick et al., 1992, J. Biol. Chem. 267:21303-21306; Melnick et al., 1994, Nature 370:373-375; Schaiff et al., 1992, J. Exp. Med. 176:657-666; Ramakrishnan et al., 1995, DNA and Cell Biol. 14:373-384). Further, expression of gp96 is induced by conditions which result in the accumulation of unfolded proteins in the endoplasmic reticulum (Kozutsumi et al., 1988, Nature 332:462-464). It has been reported that gp96 appears to have ATPase activity (Li and Srivastava, 1993, EMBO J. 12:3143-3151), but this observation has been questioned (Wearsch and Nicchitta, 1997, J. Biol. Chem. 272:5152-5156).

Inoculation with heat shock protein prepared from tumors of experimental animals has been shown to induce immune responses in a tumor-specific manner; that is to say, heat shock protein gp96 purified from a particular tumor could induce an immune response which would inhibit the growth of cells from the identical tumor of origin, but not other tumors, regardless of relatedness (Srivastava and Maki, 1991, Curr. Topics Microbiol. 167:109-123). The immunogenic portion of the tumor-isolated heat shock proteins has been attributed to immunogenic peptides bound to the heat shock proteins, and the specificity of the immunogenicity residing therein (Srivastava, 1991, Curr. Opinion Immunol. 3:654-658). This finding was supported by the screening of a peptide epitope library with gp96 (idem). Moreover, binding affinity of various peptides to heat shock proteins in vitro has been established (e.g., Flynn et al., Science 245: 385-390 (1989)).

Genes encoding heat shock proteins have not been found to exhibit tumor-specific DNA polymorphism (Srivastava and Udono, 1994, Curr. Opin. Immunol. 6:728-732). High-resolution gel electrophoresis has indicated that tumor-derived gp96 may be heterogeneous at the molecular level; evidence suggests that the source of this heterogeneity may be populations of small peptides adherent to the heat shock protein, which may number in the hundreds (Feldweg and Srivastava, 1995, Int. J. Cancer 63:310-314). Indeed, an antigenic peptide of vesicular stomatitis virus has been shown to associate with gp96 in virus infected cells (Nieland et al., 1996, Proc. Natl. Acad. Sci. U.S.A. 93:6135-6139). It has been suggested that this accumulation of peptides is related to the localization of gp96 in the endoplasmic reticulum, where it may act as a peptide acceptor and accessory to peptide loading of major histocompatability complex class I molecules (Li and Srivastava, 1993, EMBO J. 12:3143-3151; Suto and Srivastava, 1995, Science 269:1585-1588).

Heat shock proteins have been used as adjuvants to stimulate an immune response (see, for example, Edgington, 1995, Bio/Technol. 13:1442-1444; PCT Application International Publication Number WO 94/29459 by the Whitehead Institute for Biomedical Research, Richard Young, inventor, and references infra). One of the best known adjuvants, Freund's complete adjuvant, contains a mixture of heat shock proteins derived from mycobacteria (the genus of the bacterium which causes tuberculosis); Freund's complete adjuvant has been used for years to boost the immune response to non-mycobacterial antigens. A number of references suggest, inter alia, the use of isolated mycobacterial heat shock proteins for a similar purpose, including vaccination against tuberculosis itself (Lukacs et al., 1993, J. Exp. Med. 178:343-348; Lowrie et al., 1994, Vaccine 12:1537-1540; Silva and Lowrie, 1994, Immunology 82:244-248; Lowrie et al., 1995, J. Cell. Biochem. Suppl. 0(19b):220; Retzlaffet al., 1994, Infect. Immun. 62:5689-5693; PCT Application International Publication No. WO 94/11513 by the Medical Research Council, Colston et al., inventors; PCT Application International Publication No. WO 93/1771 by Biocine Sclavo Spa, Rappuoli et al., inventors).

Other references focus on the ability of heat shock proteins to naturally form associations with antigenic peptides, rather than the classical adjuvant activity (see, for example PCT Application No. PCT/US96/13233 by Sloan-Kettering Institute for Cancer Research, Rothman et al., inventors; Blachere and Srivastava, 1995, Seminars in Cancer Biology 6:349-355; PCT Application International Publication No. WO 95/24923 by Mount Sinai School of Medicine of the City University of New York, Srivastava et al., inventors). In one protocol used by Srivastava in a phase I European clinical trial, cells prepared from a surgically resected tumor were used to prepare gp96, which was then reinoculated into the same patient (Edgington, 1995, Bio/Technol. 13:1442-1444). PCT Application International Publication No. WO 95/24923 (supra) suggests that peptides in heat shock protein complexes may be isolated and then re-incorporated into heat shock protein complexes in vitro.

Although the immunogenic potential of heat shock proteins and molecular chaperones has been clearly demonstrated (reviewed by Schild H. et al., Current Opinion in Immunology (1999) 11:109 113), whereby heat shock proteins are believed to deliver bound antigens to antigen presenting cells for subsequent display on MHC class I or class II molecules (thereby generating a T cell response), many antigens do not bind sufficiently well to heat shock proteins or molecular chaperones for them to be efficiently delivered. In attempts to circumvent this limitation, heat shock proteins have been covalently joined to antigenic peptides of choice. For example, it has been reported that a synthetic peptide comprising multiple iterations of NANP (Asn Ala Asn Pro; SEQ ID NO: 2) malarial antigen, chemically crosslinked to glutaraldehyde-fixed mycobacterial heat shock proteins hsp65 or hsp70, was capable of inducing a humoral (antibody based) immune response in mice in the absence of further adjuvant; a similar effect was observed using heat shock protein from the bacterium Escherichia coli (Del Guidice, 1994, Experientia 50:1061-1066; Barrios et al., 1994, Clin. Exp. Immunol. 98:224-228; Barrios et al., 1992, Eur. J. Immunol. 22:1365-1372). Cross-linking of synthetic peptide to heat shock protein and possibly glutaraldehyde fixation were required for antibody induction (Barrios et al., 1994, Clin. Exp. Immunol. 98:229-233), and cellular immunity does not appear to be induced. In another example, Young et al., in PCT Application International Publication Number WO 94/29459, discloses fusion proteins in which an antigenic protein is joined to a heat shock protein. PCT Application No. PCT/US96/13363 (WO97/06821) by Sloan-Kettering Institute for Cancer Research, Rothman et al., inventors, describe the use of heat shock protein binding domains, coupled to an immunogenic domain of an antigen, to increase its binding to a heat shock protein and thus the delivery to the immune system for eliciting an immune response to the antigen.

Identification of a specific cell surface receptor for heat shock proteins on cells, including antigen presenting cells, has led to a further appreciation for the intracellular processing of heat shock proteins and complexes of heat shock proteins and associated antigens (Binder et al., 2000, J. Immunol. 165:2582-2587; Sondermann et al., 2000, Biol. Chem 381:1165-1174; Castellino et al., 2000, J. Exp. Med. 191:1957-1964), and recently, several previously-known cell surface receptor molecules have been identified as heat shock protein receptors, resulting in a reinterpretation of their role in the immune system. For example, a CD14-dependent pathway for hsp70 has been reported (Asea et al., 2000, Nature Medicine 6:435-442) and CD91 has also been implicated as a receptor for certain heat shock proteins (Basu et al., 2001, Immunity 14:303-313; Binder et al., 2000, Nature Immunology 1:151-155). More recently, CD36 has been proposed as a heat shock protein receptor (PCT/US01/31401; US2002/0086276A1).

It is toward the identification of specific cell surface receptor molecules for heat shock proteins, in particular for mammalian heat shock proteins, and more particularly for complexes thereof with antigens or other molecules, and modulation of such receptor activities for the purpose of altering the immune response to an immunogenic molecule associated with a heat shock protein, exploiting the interaction therebetween for targeting molecules to cells expressing or that can be induced to express such receptors, as well as to taking advantage of the cell signaling and uptake properties from the interaction of a heat shock protein-antigen complex with such a receptor therefor, that the present invention is directed.

### SUMMARY OF THE INVENTION

In a first aspect, a method is provided for enhancing the ability of a cell to bind to a mammalian heat shock protein and preferably a mammalian heat shock protein in a complex with a molecule such as an antigen, by at least exposing the cell to at least one agent capable of inducing or increasing CD40 expression by the cell. Inducing or increasing CD40 expression by a cell may be achieved, for example, by introducing a polynucleotide encoding an expressible CD40 or heat shock protein-binding fragment into the cell, or by exposing the cell to an agent that causes the expression or increased expression of CD40, such as but not limited to a calcium ionophore such as A23187 or ionomycin; to a cytokine such as IL-1alpha, TNF-alpha, IFN-gamma, IL-3 or GM-CSF; or to lipopolysaccharide (LPS). One or a combination of agents or methods may be used to achieve the aforementioned purpose, such as but not limited to introducing into the cell by any of various means an expressible vector comprising a polynucleotide encoding CD40 under control of a constitutive promoter, or an inducible promoter and concomitantly or subsequently exposing the cells to an inducer, that increases the expression of CD40 encoded by the introduced polynucleotide. In all of the embodiments herein, the expressed CD40 may be a fragment of or a sequence modification of native CD40 wherein expression of the encoding polynucleotide results in the cell exhibiting CD40-like heat shock protein-binding affinity, signaling, or internalization properties, and any combination of the foregoing.

The heat shock protein or fragment thereof whose binding to a cell is enhanced or increased by this aspect of the invention preferably is native, or a fragment of a native, mammalian hsp70 family member, such as hsp70, hsc70 or BiP, more preferably mammalian hsp70 and most preferably human hsp70. However, the invention is not so limited and is applicable to any heat shock protein, preferably mammalian, that binds to CD40. Alternately, the heat shock protein may be a modified heat shock protein or fragment thereof with enhanced affinity for CD40. In one non-limiting embodiment, the fragment is the N-terminal domain of an hsp70 that comprises the ATP-binding domain, such as but not limited to residues from about amino acid number 1-5 extending to about amino acid number 381. In another embodiment, a fragment of the N-terminal or ATP-binding domain of hsp70 may be used, the fragment of at least about 6 amino acids and capable of binding to CD40.

Preferably, the heat shock protein or CD-binding fragment thereof enhancedly bound to a cell in the aforementioned aspect of the invention, as well as in the following aspects of the invention, is provided in an association with, or complexed with, a pre-selected molecule, the pre-selected molecule either covalently or noncovalently bound to the heat shock protein. In one non-limiting embodiment, the complex may be an in-vitro prepared, non-covalent complex of a heat shock protein and a hybrid molecule, the hybrid molecule comprising a covalent conjugate between an antigen and a heat shock protein binding moiety. The heat shock protein binding moiety may be a peptide or an organic molecule, by way of non-limiting example. In another embodiment, the complex is a non-covalent association between an antigen and the heat shock protein or a fragment thereof based on the natural affinity between the antigen and the heat shock protein or fragment thereof. Such complexes may be prepared in vitro from one or more defined antigens and heat shock protein(s), or isolated from cells, tissues, or other biological materials in which such complexes are present or are made to be present. In the foregoing embodiments, the preferable heat shock proteins or fragments thereof comprise in addition to a CD40-binding domain, a peptide binding domain. In a third embodiment, the heat shock protein or CD40-binding fragment thereof is covalently bound to the pre-selected molecule, which may be or comprise an antigen or epitope. The foregoing covalent complex may be synthesized chemically or expressed recombinantly as a fusion polypeptide, and linker such as a short peptide may be interposed therebetween. In this embodiment, the heat shock protein or CD40-binding fragment may not necessarily comprise a peptide (antigen) binding domain, as the antigen is covalently bound thereto. The antigen in any of the foregoing embodiments may be, for example, a peptide, protein, carbohydrate, nucleic acid, lipid, glycoprotein, glycolipid, or a viral particle, or any combination of the foregoing. The antigen may be a tumor associated antigen or an infectious disease associated antigen, or any pathology-related antigen. Included with the heat shock protein complex and particularly with non-covalent complexes in all of the embodiments herein may be one or more additional agents that maintain or promote the non-covalent binding of the pre-selected molecule to the heat shock protein or fragment, by way of non-limiting example, adenosine diphosphate (ADP). Preferably, the formulations comprising heat shock protein and non-covalently bound antigen include ADP. Other nucleotides or nucleotide analogues, whether naturally occurring or synthetic, with the same activity of maintaining and/or promoting the binding of the pre-selected molecule to the heat shock protein may be used alone or included therewith.

The aforementioned cells may be professional phagocytes, such as antigen presenting cells, macrophages, B cells or neutrophils; the selection of antigen presenting cells including dendritic cells such as myeloid dendritic cells or lymphoid dendritic cells. The cells may be non-professional phagocytes, such as endothelial cells, epithelial cells, or fibroblasts, by way of non-limiting example. They may be derived from or present in an animal, preferably a mammalian animal, more preferably a human, or obtained from tissue culture.

The foregoing method may be carried out by exposing the aforementioned cells to the at least one such agent either in vitro, in vivo, or ex vivo, or any combination of the foregoing. Cells exposed in vitro or ex vivo may then be administered to a mammal or returned to a mammal, respectively. The cells may be exposed to the at least one agent ex vivo or in vitro, and then after introduction into an mammal, the mammal may be administered the same or a different agent to further enhance the expression of CD40 by the cells. These protocols are merely exemplary and non-limiting. Mammals as described herein preferably are humans, but the invention is also inclusive of primates, domestic, livestock and companion mammalian animals, among other mammals and is not intended to be at all limiting.

In a non-limiting in-vitro embodiment of this aspect of the invention, cells derived from tissue culture, or from an animal such as a mammal other than the mammal into which the cells will be eventually administered, or from any source other than the recipient, are exposed to an agent capable of inducing or increasing expression of CD40, as hereinbefore described. After exposure, the cells optionally may be exposed to additional agents, among which may be at least one pre-selected or mixture of molecules associated with a heat shock protein, prepared in vitro or isolated from cells or tissues, as will be described in detail below. The mixture may be administered to a mammalian patient; optionally, the cells may be washed prior to administration such that only the cells and any molecules bound thereto are administered. After return to the patient, the patient optionally may be administered a complex of a heat shock protein and an antigen. Thus, the patient may be administered the heat shock protein, preferably a complex with an antigen or other molecule, before, during or after administration of the cells, or any combination of the foregoing.

In a non-limiting ex-vivo embodiment, phagocytic cells may be removed from a mammal, exposed to at least one agent capable of inducing or increasing expression of CD40. After exposure, the cells optionally may be exposed to additional agents, including a pre-selected molecule associated with a heat shock protein or fragment thereof, as will be described hereinbelow. The cells then may be returned to the mammalian patient; optionally, the cells may be washed prior to administration such that only cells and molecules bound thereto are administered. The patient may be administered the heat shock protein before, during or after administration of the cells, or any combination of the foregoing.

In a non-limiting in-vivo embodiment, at least one agent capable of inducing or increasing CD40 expression in phagocytic cells may be administered to a mammalian patient, optionally in combination before, during or afterwards, with a heat shock protein, or CD40-binding fragment thereof, preferably either of the foregoing complexed with a molecule, preferably a mammalian heat shock protein, more preferably hsp70, and most preferably human hsp70, as described hereinabove. The administration may be through a parenteral or oral route, and by way of non-limiting example, topically or systemically administered. Administration preferably is followed by administration of at least one pre-selected molecule in association with a heat shock protein or fragment thereof.

In a second aspect, a method is provided for delivering or increasing the delivery of a heat shock protein or a CD40-binding fragment thereof, preferably in a complex with a molecule further which preferably is an antigen, into a cell, comprising exposing the cell to at least one agent capable of inducing or increasing CD40 expression by the cell, as mentioned hereinabove, followed by exposing the cell to a mammalian heat shock protein or CD40-binding fragment thereof, preferably a human heat shock protein, more preferably an hsp70 and most preferably human hsp70. A preferred fragment comprises the N-terminal or ATP-binding (ATPase) domain of hsp70, or a CD40-binding portion thereof, as mentioned herein above. Inducing or increasing CD40 expression by a cell may be achieved, for example, by introducing by any of several means a polynucleotide encoding an expressible CD40 into the cell, or by exposing the cell to an agent that causes the expression or increased expression of CD40, such as by exposure to one or more calcium ionophores, cytokines or LPS as mentioned above. As noted above, combinations of agents may be used to achieve the induction or increase in CD40 expression.

The heat shock protein may be, by way of non-limiting example, gp96, hsp70, hsp90, hsc70, hsp110, or BiP, or any members of families of heat shock proteins that include any of the aforementioned members. Mammalian heat shock proteins are preferred, mammalian hsp70 is more preferred, and human hsp70 most preferred. Preferably, the heat shock protein or fragment thereof in the aforementioned and following aspects of the invention is provided in an association with or in a complex with a pre-selected molecule, such as an antigen or epitope thereof, the pre-selected molecule either covalently or noncovalently bound to the heat shock protein. Embodiments of such complexes are described herein above: where the complex is non-covalent, the CD40-binding heat shock protein or fragment thereof preferably comprises a pre-selected molecule binding domain; where the complex is covalent, the heat shock protein or CD40-binding fragment thereof need not necessarily naturally bind to the pre-selected molecule. In one non-limiting embodiment, the complex may be a non-covalent complex of a heat shock protein or fragment thereof and a hybrid molecule, the hybrid molecule comprising a covalent conjugate between a molecule such as an antigen, preferably an epitope and most preferably a peptide epitope, and a heat shock protein binding moiety (also referred to herein as a heat shock protein binding domain). The heat shock protein binding moiety may be a peptide or an organic molecule, by way of non-limiting example. In another embodiment, the complex is a non-covalent association between the molecule such as an antigen, and the heat shock protein or a fragment thereof based on the natural affinity between the heat shock protein or fragment and the antigen or another molecule. Such complexes may be prepared in vitro or isolated from cells, tissues or other biological materials containing them or that may be made to contain them. In a third embodiment, the heat shock protein or CD40-binding fragment thereof is covalently bound to the pre-selected molecule, which may be or comprise an antigen or epitope thereof. The foregoing covalent complex may be synthesized chemically or if a polypeptide expressed recombinantly as a fusion polypeptide, and optionally a linker such as a short peptide may be interposed therebetween. The antigen in any of the foregoing embodiments may be, for example, a peptide, protein, carbohydrate, nucleic acid, lipid, glycoprotein, glycolipid, or a viral particle, or any combination of the foregoing. The antigen may be a tumor associated antigen or an infectious disease associated antigen, or any pathology-related antigen. Included with the heat shock protein complex and particularly with non-covalent complexes in any of the embodiments herein may be one or more additional agents that maintain or promote the non-covalent binding or affinity of the selected molecule to or for the heat shock protein, by way of non-limiting example, adenosine diphosphate (ADP). Preferably, the formulations comprising heat shock protein and non-covalently bound antigen include ADP. Other nucleotides or nucleotide analogues, whether naturally occurring or synthetic, with the same activity of maintaining and/or promoting the binding of the pre-selected molecule to the heat shock protein may be used alone or included therewith.

In the foregoing example, the heat shock protein is preferably mammalian, more preferably human and most preferably human hsp70. Alternately, the heat shock protein may be a fragment or portion of a heat shock protein molecule that binds to CD40. In one non-limiting embodiment, the fragment is the N-terminal domain of hsp70 that comprises the ATP-binding domain, such as but not limited to residues from about amino acid 1-5 and extending to about amino acid number 381. In another embodiment, a fragment of the N-terminal or ATP-binding domain of hsp70 may be used, the fragment capable of binding to CD40. One of skill in the art will readily identify a fragment of hsp70 capable of binding to CD40. Such fragments are preferably at least six amino acids. In the foregoing instances, the heat shock protein fragment may be associated preferably covalently or, if the fragment has a binding affinity, non-covalently with or be a complex with a pre-selected molecule such as an antigen, as described above. The antigen may be covalently bound to the fragment, such as by chemical means or by synthesis as a fusion polypeptide. A linker peptide may optionally be provided therebetween. The invention also embraces polynucleotide sequences including degenerate sequences that encode a polypeptide comprising a CD40-binding fragment of a heat shock protein and a peptide or protein antigen desirably delivered by the method of the present invention or for any other purpose. In another embodiment, the pre-selected molecule may be non-covalently associated with the heat shock protein fragment.

The aforementioned cells may be professional phagocytes, such as antigen presenting cells, macrophages, B cells or neutrophils, the selection of antigen presenting cells including dendritic cells such as myeloid dendritic cells or lymphoid dendritic cells; or the cells may be non-professional phagocytes, such as endothelial cells, epithelial cells, or fibroblasts, by way of non-limiting examples. They may be derived from a mammalian animal, preferably a human, or from tissue culture.

The foregoing method may be carried out by exposing the cells to at least one of the aforementioned CD40-inducing, CD40-expressing or CD40-upregulating agents and a heat shock protein or fragment thereof, preferably in association with a pre-selected molecule, either in vitro, in vivo, or ex vivo, or in any combination of the foregoing. Cells exposed in vitro or ex vivo may then be administered to a mammal or returned to a mammal, respectively. Exposure to the heat shock protein or fragment thereof and preferably in association with a molecule may also be in vitro, in vivo or ex vivo, yet the exposure to the agent for increasing CD40 expression and exposure to a heat shock protein-molecule complex may be independently selected and carried out. Preferably, the heat shock protein is a complex of a heat shock protein or fragment thereof and a pre-selected molecule, as described above. In the foregoing example, the heat shock protein is preferably mammalian, more preferably human and most preferably human hsp70.

Thus, in a preferred embodiment, a method is provided for delivering or increasing the delivery of a mammalian heat shock protein or fragment thereof and most preferably human hsp70 to a cell, comprising exposing the cell to at least one agent capable of inducing or increasing CD40 expression by the cell, preceded by, concurrently or followed by exposing the cell to a heat shock protein. The heat shock protein is preferably complexed or associated with another molecule desirably delivered by the methods of the present invention, as hereinbefore described. Inducing or increasing CD40 expression by a cell may be achieved, for example, by introducing a vector encoding an expressible CD40 polynucleotide into the cell, or by exposing the cell to an agent that causes the expression or increased expression of CD40, such as by exposure to one or more calcium ionophores, cytokines or LPS, by way of non-limiting example. As noted above, combinations of agents may be used to achieve the induction of or increase in CD40 expression.

In a non-limiting in-vitro embodiment, cells derived from tissue culture, or from a mammal other than the mammal into which the cells will be administered, or from any other source than the recipient, are exposed to at least one agent capable of inducing or increasing expression of CD40. After exposure, the cells are exposed to an antigen or other pre-selected molecule associated with a heat shock protein or a CD40-binding fragment thereof, as hereinbefore described. Such exposure may be in vitro, or in vivo after the cells are delivered to the recipient. The heat shock protein is preferably mammalian, more preferably human and most preferably human hsp70. Additional agents such as ADP may be included. Other nucleotides or nucleotide analogues, whether naturally occurring or synthetic, with the same activity of maintaining and/or promoting the non-covalent binding of the pre-selected molecule to the heat shock protein may be used alone or included therewith. The mixture may be administered to a mammalian patient; optionally, the cells may be washed prior to administration. Heat shock protein may be administered to the recipient before or after administration of cells.

In a non-limiting ex-vivo embodiment, phagocytic cells may be removed from a mammal, exposed to at least one agent capable of inducing or increasing expression of CD40. After exposure, the cells are exposed to a pre-selected molecule associated with a heat shock protein or CD40-binding fragment thereof, preferably with a molecule complexed thereto as described hereinabove. The cells then may be returned to the mammalian patient; optionally, the cells may be washed prior to administration such that only cells and bound molecules are administered. Other agents may be included with the cells when administered. In an alternate embodiment, the mammalian patient may be administered heat shock protein or complexes as described herein prior to or after administration of the cells.

In a non-limiting in-vivo embodiment, at least one agent capable of inducing or increasing CD40 expression in phagocytic cells may be administered to a mammalian patient, the administration temporally associated with administration of a heat shock protein preferably in a complex with a molecule as described herein. Administration may be concurrent, or either one may be given prior to the other. In a preferred embodiment, the at least one agent capable of inducing or increasing CD40 expression is administered to the mammal first, and the heat shock protein administered after the expression of CD40 has increased. The heat shock protein is preferably a complex of a heat shock protein or CD40-binding fragment thereof and a pre-selected molecule, and more preferably an antigen or immunogen. The heat shock protein is preferably mammalian, and more preferably human. Human hsp70 is most preferred. Administration may be parenteral or oral, topical or systemic, and preferably subcutaneous or intradermal, by way of non-limiting examples, as described herein.

In the aforementioned aspects of the invention directed towards increasing the ability of a heat shock protein, preferably in a complex with a molecule, to bind to a cell by inducing or increasing CD40 expression, the attendant increased binding of the heat shock protein to CD40 is useful for a variety of purposes. By way of non-limiting examples, the increased binding of the heat shock protein to CD40 is useful for providing for or increasing the delivery or uptake into cells of molecules such as antigens or epitopes thereof complexed covalently or non-covalently with heat shock proteins. Such increased uptake is desirable for a number of purposes. In one example, increased uptake results in facilitating increased processing of the antigen and attendant antigen presentation for the induction of an immune response to the antigen. In another non-limiting example, binding of heat shock protein to CD40 on cells that have been induced to express or having increased expression of CD40 results in cell signaling, which is useful, for example, for modulating maturation of antigen presenting cells such as dendritic cells, such that with the combination of the antigen, a more robust immune response is generated. Under certain conditions tolerization to an antigen may be induced by the methods of the invention, for the purpose of decreasing or preventing the induction of an immune response to an autoimmune antigen or a transplant antigen or allergen. Increased uptake of heat shock protein or a fragment thereof may also be used to deliver polynucleotides such as expressible sequences and antisense sequences, for the purpose of altering the genotype of the cell, with or without altering its phenotype. These uses of the enhanced heat shock protein binding properties of cells made possible by the methods of the invention are merely exemplary and not intended at all to be limiting. In the foregoing example, the heat shock protein is preferably mammalian, more preferably human and most preferably human hsp70.

The selection of cell type, heat shock protein or fragment thereof and complexes thereof with at least one associated molecule, and cells, are as described herein. As noted above, such increased uptake may be for the purpose of inducing an immune response to at least one molecule, or to delivery of polynucleotides such as expressible sequences, or antisense sequences, for the purpose of altering the genotype and possibly the phenotype of the cell. These uses are merely exemplary and non-limiting.

Thus, in yet another embodiment of this aspect of the invention, a method is provided for enhancing the development in a mammal of an immune response toward one or more antigens by inducing or increasing CD40 expression in phagocytes of a mammal, followed by exposing the phagocytes to a complex comprising a heat shock protein and at least one antigen. This modulation of CD40 expression may be carried out in vitro, in vivo or ex vivo, or combinations thereof, and independently, the exposure to the heat shock protein complex may be in vitro, ex vivo or in vivo. Depending on the amount of heat shock protein-antigen complex used, and the presence or absence of co-stimulatory molecules or cells, a cytolytic or humoral immune response, or both, may be elicited, or tolerance to the antigen may be induced. Such conditions are readily determinable by one skilled in the art. In the foregoing example, the heat shock protein associated with the antigen is preferably mammalian, more preferably human and is most preferably human hsp70.

The phagocytes may be exposed to at least one agent capable of inducing or increasing CD40 expression. The agent may be a vector comprising a polynucleotide encoding CD40, or it may be a calcium ionophore, cytokine, LPS, or any other agent such as but not limited to any mentioned hereinabove capable of increasing CD40 expression. Examples of selections of heat shock proteins are as described above.

Thus, in one in-vitro embodiment of this aspect of the invention, at least the following steps may be carried out:

i) obtaining a sample of phagocytes from cell culture or from an animal, preferably a mammal other than the recipient mammal;

ii) exposing the phagocytes to at least one agent capable of inducing or increasing CD40 expression by the phagocytes;

iii) exposing the phagocytes of step (ii) to a effective immune response inducing amount of a complex containing at least a heat shock protein or a CD40-binding fragment thereof, and an antigen; and

iv) administering the phagocytes to the mammal.

The phagocytes optionally may be washed after step (ii) or step (iii), or both, before administering to the mammal. In an alternate embodiment, step (iii) is carried out in vitro and exposure to the complex is provided in vivo, by administering the complex to the mammal before or after the phagocytes are administered to the mammal. Of course, both in vitro and in vivo exposure to complexes may be performed.

In one ex-vivo embodiment, the invention may be carried out by following at least the steps of:

i) removing a sample of phagocytes from a mammal;

ii) exposing the phagocytes to at least one agent capable of inducing or increasing CD40 expression by the phagocytes;

iii) exposing the phagocytes of step (ii) to a effective immune response inducing amount of a complex containing at least a heat shock protein or CD40-binding fragment thereof and the antigen; and

iv) returning the phagocytes to the mammal.

The phagocytes optionally may be washed after step (ii) or step (iii), or both, before administering the cells to the mammal. In an alternate embodiment, the exposure of the complex is provided in vivo, by administering complexes to the mammal prior to or after returning the phagocytes to the mammal. Of course, both ex vivo and in vivo exposure to complexes may be performed.

An in vivo embodiment of this aspect of the invention may be carried out by at least the steps of:

i) administering to the animal an agent capable of increasing CD40 expression by phagocytic cells in the animal; and

ii) administering to the animal prior to, concurrently, afterwards, or any combination thereof, an effective immune response inducing amount of a conjugate of a mammalian heat shock protein or CD40-binding fragment thereof and the antigen.

In the foregoing examples, the heat shock protein in the complex is preferably mammalian, more preferably human and most preferably human hsp70. CD40-binding fragments of heat shock proteins are also described herein above.

In any and all of the in vivo procedures and methods of the present invention, the mammal may be any mammal, preferably a human mammal but also a domesticated, companion or livestock animal. The phagocytes may be professional phagocytes, such as an antigen presenting cell, macrophage, B cell or neutrophil, the selection of antigen presenting cells including dendritic cells such as myeloid dendritic cells or lymphoid dendritic cells; or non-professional phagocytes, such as an endothelial cell, an epithelial cell, or a fibroblast, by way of non-limiting example. The at least one agent capable of increasing expression of CD40 by the aforementioned cells may be a vector comprising a polynucleotide encoding CD40, or, for example, a calcium ionophore, cytokine, or LPS, as mentioned above.

The effective immune response-inducing amount of a complex containing at least a heat shock protein or CD40-binding fragment thereof and an antigen may be a covalent or noncovalent complex comprising the antigen or an immunogenic fragment thereof and the heat shock protein or fragment thereof, as hereinbefore described. The heat shock protein is preferably mammalian, more preferably human and most preferably human hsp70, but it is not so limiting. In one non-limiting embodiment, the fragment is the N-terminal domain of hsp70 that comprises the ATP-binding domain, such as but not limited to residues from about amino acid number 1-5 and extending to about amino acid number 381 of human hsp70. In another embodiment, a fragment of the N-terminal or ATP-binding domain of hsp70 may be used, the fragment capable of binding to CD40. One of skill in the art will readily identify other fragments of hsp70 capable of binding to CD40, by methods such as but not limited to those described hereinbelow. Such fragments are preferably covalently bound to the antigen as mentioned herein. The complex may be a non-covalent complex of a heat shock protein and a hybrid antigen, the hybrid antigen comprising a covalent conjugate between the selected antigen or an immunogenic fragment thereof and a heat shock protein binding moiety. The heat shock protein binding moiety may be a peptide or an organic molecule. The complex may be a non-covalent complex of a heat shock protein and an antigen based on the natural affinity between the heat shock protein and the antigen, the complexes prepared in vitro or isolated from cells, tissues, or other biological material in which such complexes are present or can be made to be present. One or more additional agents may be included with the above-mentioned complexes, and in particular the non-covalent complexes, that maintain or promote the non-covalent binding of the pre-selected molecule to the heat shock protein, such as ADP. Other nucleotides or nucleotide analogues, whether naturally occurring or synthetic, with the same activity of maintaining and/or promoting the non-covalent binding of the pre-selected molecule to the heat shock protein may be used alone or included therewith.

The antigen preferably may be an infectious disease or tumor antigen, if an enhanced immune response is desired, but it is not so limiting. Such antigens may be, by way of non-limiting examples, peptides, proteins, carbohydrates, lipid, glycoproteins, glycolipids, and nucleic acids. Induction of tolerance to autoimmune antigens, transplant antigens and allergens, among others, is also desirable, and such antigens are fully embraced herein. In this and any of the aspects of the invention herein, the heat shock protein and associated molecules or antigens maybe prepared in vitro as described, or such complexes or associations may be isolated from cells, such as diseased cells, which contain complexes of heat shock proteins and antigens naturally bound thereto. In a further embodiment, non-diseased cells may be modified to contain such complexes by introducing DNA encoding heat shock proteins and/or DNA encoding antigens thereinto, and subsequently isolating useful complexes for the purposes herein.

In addition to the foregoing methods for inducing or enhancing binding and uptake of heat shock proteins and associated molecules to cells, such as to induce an immune response to an antigen complexed with the heat shock protein, in another embodiment of the invention, the binding and uptake of heat shock proteins to cells may be reduced or inhibited by interfering with the binding between a heat shock protein and CD40. Such inhibition may be carried out in vitro, ex vivo, or in vivo, and any combination of the foregoing. The various cell types, heat shock protein complexes, and other common features of this aspect of the invention are as described above.

Thus, in still yet a further aspect of the invention, a method is provided for decreasing the binding, signaling and/or uptake of a heat shock protein or CD40-binding fragment thereof by a cell expressing CD40 or a cell induced to express CD40 by at least exposing said cell in vitro or ex vivo to an agent capable of interfering with the binding of the heat shock protein to CD40 expressed by the cell. The agent may be, for example, a CD40 binding partner that does not induce signaling or uptake, such as an non-agonistic antibody or a ligand of CD40. Preferably, the ligand is a heat shock protein, a fragment of a heat shock protein, or CD40L (CD40 ligand or CD154). An exemplary non-agonistic antiCD40 monoclonal antibody is 5D12.

The phagocytes may be professional phagocytes, such as an antigen presenting cell, macrophage, B cell or neutrophil, the selection of antigen presenting cells including dendritic cells such as myeloid dendritic cells or lymphoid dendritic cells; or non-professional phagocytes, such as an endothelial cell, an epithelial cell, or a fibroblast, by way of non-limiting example.

Thus, in still yet another aspect of the invention, a method for decreasing the development in a mammal of an immune response is provided by carrying out at least the steps of:

i) removing a sample of phagocytes from the mammal:

ii) exposing the phagocytes to an agent capable of decreasing CD40 expression by the cells; and

iii) returning the phagocytes to the mammal.

The agent may be a CD40 antisense oligonucleotide. The phagocytes may be as described hereinabove.

The present inventors found surprisingly and unexpectedly that CD40 binds a mammalian heat shock protein, and moreover that it binds via the N-terminal or ATP-binding (ATPase) domain of the hsp70. Thus, the mammalian heat shock protein was discovered to be able to exhibit two important functions: 1) bind a peptide through the peptide-binding pocket through the C-terminal domain and also 2) bind CD40 through the N-terminal domain. Therefore, the heat shock protein can both bind to the peptide and bind to a cell via CD40. More particularly, the inventors discovered that the binding of mammalian heat shock protein to CD40 is enhanced when peptide is bound to the peptide-binding domain of heat shock protein, and additionally, that binding occurs between CD40 and the N-terminal or ATP-binding domain of hsp70 alone. These findings provide agents and methods for targeting pre-selected molecules to cells that express or may be induced to express CD40. Thus, in addition to the above-described methods and uses based hereon, in still yet a further aspect of the invention, methods are provided for targeting molecules, such as but not limited to epitopes, antigens, antigenic fragments and otherwise immunogenic peptides, to a cell that expresses CD40 or can be modified to express CD40, by utilizing a complex or association between the molecule and a mammalian heat shock protein, or preferably to a fragment of a mammalian heat shock protein that specifically binds to CD40. In one embodiment, the fragment comprises the N-terminal domain of hsp70, which is the nucleotide-binding (ATPase) domain of hsp70. In another embodiment, the fragment comprises a CD40-binding domain or fragment of hsp70, such as but not limited to a fragment of from about amino acids 1-5 and extending to about amino acid 381 of mammalian hsp70, preferably human hsp70. In another embodiment, a CD40-binding fragment of the N-terminal domain of hsp70 of at least 6 amino acids is provided as such a targeting molecule. Such complexes may be covalent or non-covalent complexes, and may comprise other components in order to effectively deliver a pre-selected molecule to a CD40-expressing cell by taking advantage of the newly described affinity between CD40 and a mammalian heat shock protein. Such methods may optionally include modulation of CD40 levels on cells before exposure as hereinabove described. Moreover, such targeting may be for purposes other than antigen delivery, such as intracellular delivery of antisense oligonucleotides, chemotherapeutic agents or modulators of the physiology of CD40-expressing cells.

Molecules targeted for CD40 binding can be derived from fragments of hsp70 or other heat shock proteins by identifying CD40-binding fragments through screening, or they may be identified based on first identifying sites of interaction between CD40 and a heat shock protein, which may be performed by x-ray crystallographic studies, site-directed mutagenesis, photo-cross-linking, and other means known to the skilled artisan, including molecular modeling. Fragments of heat shock proteins or small organic molecules with CD40 binding activity identified by their ability to interfere with the interaction between CD40 and a heat shock protein are fully embraced herein.

In one embodiment, the targeting means are peptide fragments of heat shock proteins, including modified heat shock proteins and heat shock protein fragments that are modified for enhanced binding to CD40, and may also optionally include modifications that enhance non-covalent binding of the heat shock protein or fragment to a peptide, should such fragments retain the latter binding activity and thus non-covalently bind to both a peptide and CD40. Such modifications may be made in the N-terminal domain of hsp70, which is the nucleotide-binding domain of hsp70, or the corresponding region of other heat shock proteins. Such modifications to enhance CD40 binding may be made in the region of the hsp70 molecule at one or more amino acid residues between about amino acid 1-5 extending to about amino acid 381. Such modifications may include, but are not limited to, amino acid substitutions, deletions, insertions, chemical derivatization of one or more amino acids, and inclusion of non-naturally-occurring amino acids such as but not limited to one or more D-amino acids. Such altered heat shock proteins or fragments may be expressed recombinantly, and-if necessary, modified afterwards, or they may be prepared synthetically by standard peptide synthesis methods and any post-synthetic modification required. The present invention embraces polynucleotide sequences including degenerate sequences that encode such altered heat shock proteins, muteins, and any that also comprise an antigenic peptide or protein in the same polypeptide chain. The heat shock proteins or fragments thereof which have modifications in the amino acid chain of the molecule are referred to herein as heat shock protein muteins or heat shock protein fragment muteins, representing the presence of one or more mutations in the otherwise wild-type amino acid sequence thereof, and the ability to express the muteins recombinantly.

In addition, the present invention is directed to screening for such CD40-targeting agents including but not limited to fragments of mammalian heat shock proteins, by using the herein-discovered affinity between CD40 and the N-domain of hsp70 as an interacting ligand-receptor pair, whose extent of interaction may be monitored for the activity of agents that may increase or compete competitively or noncompetitively for binding to CD40. The native heat shock protein molecule or a CD40-binding fragment may be used, such as those mentioned herein; the full-length CD40 molecule or a heat shock protein binding fragment thereof such as the exoplasmic domain may be used, as considerations of the solubilities of the binding partners may be exploited for a particular assay format, whether entirely liquid phase, solid-liquid phase, etc. Any of various automated, semi-automated or manual screening procedures including high-throughput screening may be established using the basic interaction between the aforementioned binding partners to identify potentially therapeutically useful agents. Such agents may be used as CD40 targeting agents as described above, for any or all of the uses that heat shock proteins of CD40-binding fragments thereof may be used in the many foregoing aspects of the invention, or as means to agonize or antagonize CD40 for immunological modulation or other purposes that will readily be evident by one of skill in the art. Agonizing CD40 is known to have immunostimulatory properties and may be used in conjunction with vaccination or other immune response-inducing procedures to elicit and enhanced immune response; alternatively, antagonists of CD40 such as by use of certain monoclonal antibodies such as 5D12 are useful for inhibiting or down-regulating an immune response, such as may occur in autoimmune disease or xenotransplant. The compounds identified by the methods of the invention based on modulating the interaction between CD40 and a mammalian heat shock protein embrace these exemplary uses as well as others that will be readily evident.

Thus, small molecule compounds that modulate the interaction between a heat shock protein and CD40, as well as CD40 agonists and antagonists, may be identified by screening activity through assays as described above, as well as rationally designed or developed based on the modeling of the interacting sites between heat shock protein and more particularly human hsp70, and CD40, as mentioned above. Such compounds identified thereby are fully embraced herein.

Complexes between the aforementioned heat shock proteins, muteins, fragments, mutein fragments or modifications thereof and the preselected molecule to be targeted to CD40 may be prepared by covalent binding of the heat shock protein or fragment and the preselected molecule, or by non-covalent association where such molecules have an affinity. In the instance of covalent association, the two members of the complex may be covalently cross-linked using a cross-linking agent such as a carbodiimide, a homobifunctional or heterobifunctional cross-linking agent, or where both members comprise amino acid chains, they may be prepared by co-linear synthesis (e.g., solid phase peptide synthesis) or expressed as a single fusion or single-chain polypeptide, optionally with a peptide linker therebetween, by recombinant means. The linking of the members will maintain the affinity of the heat shock protein or fragment portion of the complex for CD40. Other components may be included in a formulation of such a targeted preselected molecule in order to effectively deliver it to a CD40-expressing cell by taking advantage of the newly described affinity between CD40 and a heat shock protein.

As mentioned above, preferably, the pre-selected molecule is an antigen or immunogen desirably delivered for the purpose of antigen processing, presentation and an attendant immune response thereto, whether an elicited humoral and/or cellular immune response, or tolerizing of the immune system thereto. More preferably, the preselected molecule is or contains within its sequence a HLA Class I or HLA Class II peptide (epitope). The heat shock protein is preferably mammalian, more preferably human and most preferably human hsp70, but may be any heat shock protein that binds or has binding affinity for CD40, preferably a mammalian heat shock protein. In a preferred embodiment, the aforementioned method utilizes a covalent conjugate between an antigen or immunogen and a CD40-binding fragment or CD40-binding-enhancing fragment of a heat shock protein, more preferably, hsp70, and most preferably human hsp70. Readily-cleavable sequences that may be attacked by cellular proteinases to produce the Class I or Class II peptide may be included. In another embodiment, in the instance where the antigen or immunogen is a protein or peptide, the conjugate is a recombinantly-expressible fusion polypeptide between a CD40-binding fragment of a heat shock protein and the antigen or immunogen. The cell may be induced or otherwise modified as described herein to induce or increase expression of CD40, and such means to increase in expression in combination together with exposing the cells to an antigen complexed with a CD40-binding fragment of a heat shock protein is also embraced herein. Moreover, any of the foregoing methods in which an antigen is desirably delivered to a cell expressing CD40 or modified to express CD40 may be carried out using the aforementioned conjugates between the antigen and the CD40-binding portion of the heat shock protein, to effect delivery.

As noted above, the pre-selected molecule for delivery by association with a heat shock protein by methods of the invention may be one or more defined molecules prepared in vitro from one or more pre-selected molecules such as antigens, and one or more heat shock proteins. Alternatively, the molecules to be delivered via CD40 by the methods of the invention may be undefined but isolated as naturally-occurring complexes of endogenous proteins or peptides and heat shock proteins isolated from cells or another biological material. A preferred source for therapeutics purposes is diseased cells which comprise immunogenic complexes between heat shock proteins and antigens capable of eliciting an immune response against the disease. In another embodiment, normal cells or tissues or those not comprising useful complexes or either one or both components thereof may be modified to express the needed component or components, such that the modified cells may be useful sources for the aforementioned complexes. Thus, cells may be transfected or otherwise modified to express one or more antigens or one or more heat shock proteins. Such modification may entail genetic modification and/or altering the growth or environmental conditions of the cells to effect the appearance of the desired complexes.

In a similar fashion to those methods described above, in vitro, ex vivo and in vivo methods may be used. The selection of pre-selected molecules to be delivered via CD40, including antigens, expressible polynucleotides, antisense oligonucleotides, etc., are embraced herein.

Thus, the aforementioned method for enhancing the development in a mammal of an immune response toward a pre-selected antigen may be provided in an ex-vivo protocol by carrying out at least the steps of:

i) removing a sample of CD40-expressing phagocytes from the mammal;

i) exposing the phagocytes to an effective immune-response-inducing amount of a complex of a CD40-binding portion of a mammalian heat shock protein thereof and the antigen; and

iii) returning the phagocytes to the mammal.

The foregoing methods may be enhanced by first increasing expression of CD40 in certain cells by the methods described herein, in vivo prior to obtaining the sample, or in vitro before exposure to the complex. Thus, the aforementioned method for enhancing the development in a mammal of an immune response toward a pre-selected antigen may be provided by carrying out at least the steps of:

i) removing a sample of phagocytes from the mammal;

ii) exposing the phagocytes to an agent capable of inducing or increasing CD40 expression by the phagocytes;

iii) exposing the phagocytes of step (ii) to a effective immune response inducing amount of a complex of a mammalian heat shock protein or CD40-binding portion thereof and the antigen; and

iv) returning the phagocytes to the mammal; or by the following steps:

i) administering to a mammal an agent capable of inducing or increasing CD40 expression in phagocytes in said mammal;

ii) removing a sample of phagocytes from the mammal;

iii) exposing the phagocytes of step (ii) to a effective immune response inducing amount of a complex of a mammalian heat shock protein or CD40-binding portion thereof and the antigen; and

iv) returning the phagocytes to the mammal.

The foregoing method may also be carried out fully in vivo by both administering the CD40-upregulating agent to the mammal and then concurrently or subsequently administering the complexes. In another embodiment, methods are provided for enhancing the development in a mammal of tolerance to a pre-selected antigen may be achieved by carrying out the steps of:

i) removing a sample of phagocytes from said mammal:

ii) exposing said phagocytes to an agent capable of increasing CD40 expression by said cell;

iii) exposing said phagocytes of step (ii) to a effective immune response tolerizing amount of a complex of a mammalian heat shock protein or CD40-binding portion thereof and the antigen; and

iv) returning said phagocytes to the mammal;

or, as in the previous method, administering the CD40 expression increasing agent to the mammal before harvesting phagocytes for ex-vivo exposure to the complex.

Thus, the foregoing invention directed to a method for enhancing the development in a mammal of an immune response toward a pre-selected antigen may be achieved in vivo by at least the steps of:

i) administering to the animal an agent capable of increasing CD40 expression by phagocytic cells in the animal; and

ii) administering to the animal an effective immune response enhancing amount of a complex of a mammalian heat shock protein or CD40-binding portion thereof and the antigen.

In the foregoing and prior examples, the order of administering cells to a mammal and that of administering a heat shock protein complex to cells in vitro or ex vivo or to the mammal in vivo may be altered without deviating from the spirit of the present invention.

In the aforementioned procedure, co-stimulatory molecules or cells also may be administered to enhance the resulting immune response.

In the foregoing aspect of the invention, a conjugate of the invention may comprise an antigen or other pre-selected molecule covalently conjugated to the N-terminal domain of hsp70, more preferably a portion of the hsp70 molecule extending from about amino acid 1-5 to about amino acid 381, and most preferably from amino acid to amino acid 381 of human hsp70 (SEQ ID NO: 1). Alternatively, smaller fragments of hsp70 at least about six amino acids in length which are readily identifiable as CD40-binding fragments may be used as well. Exemplary portions and fragments of hsp70 are described hereinabove.

Compositions comprising both a CD40 expression upregulator and a complex or conjugate as described above are also embraced herein. The CD40 expression upregulator may be a calcium ionophore, cytokine or LPS as described above, and the complex or conjugate a molecule comprising a pre-selected molecule for delivery and an heat shock protein or CD40-binding portion thereof or a mutein thereof.

The present inventors have identified the interaction between CD40 and heat shock protein, and in particular a heat shock protein loaded with a peptide, as capable of binding, signaling via p38 the NF-kappaB pathway, and internalization of the heat shock protein-antigen complex. The induction of signaling by heat shock protein alone indicates that heat shock protein alone can provide T cell help, and these data taken together indicate that heat shock protein-antigen complexes alone can both deliver antigen to antigen presenting cells and also trigger signal transduction for the induction of the immune response. Thus, the foregoing procedures for the induction of an immune response do not necessarily rely on another source of T cell help, such as one or more adjuvants, T cell helper epitopes (e.g., tetanus toxoid), CpG or other immunomodulatory sequences, CD40L or agonistic anti-CD40 antibodies, cytokines, or other adjuvants, although such molecules providing T cell help may also be included to even further enhance the response. The ability of heat shock protein to provide such help may be advantageously exploited in several aspects.

Thus, in yet another aspect of the invention, a means for inducing the maturation of dendritic cells or other antigen presenting cells is provided by utilizing a CD40-binding fragment or a CD40-binding-enhanced fragment of a mammalian heat shock protein as a ligand for CD40 and thus a maturation signal for CD40-expressing antigen presenting cells. A mammalian heat shock protein fragment which binds CD40 and induces CD40-mediated signal transduction is preferred. The heat shock protein is preferably human and more preferably human hsp70. The fragment may be the N-terminal or ATP-binding domain of hsp70, more preferably a polypeptide from about amino acid 1-5 extending to about amino acid 381 of human hsp70, and most preferably a polypeptide having amino acid 5 to amino acid 381 of hsp70 (SEQ ID NO: 1). Muteins of the aforementioned molecules are also embraced herein, with enhanced signaling. Such signaling-enhanced heat shock protein fragments or muteins may or may not be the same as those optimized binding to CD40 for the purpose of delivering a covalently-bound or non-covalently-associated antigen. The skilled artisan can readily assay signaling and antigen delivery and determine the independent activities of such fragments and muteins, such as by the methods described in the Examples herein or those modified for high-throughput screening, for example. Preferably, an optimized heat shock protein fragment for antigen delivery for induction of an immune response is enhanced for both signaling and antigen delivery, and may have one or more modified amino acids (or insertions, deletions, alterations, etc.) in the N-terminal domain and one or more modified amino acids (or insertions, deletions, alterations, etc.) in the C-terminal domain. In other instances, for example, for non-immunologic delivery or for decreasing the immune response, an enhanced antigen carrier molecule with reduced signaling activity may be desired, and such heat shock protein fragments with independently variable signaling and delivery activities are fully embraced by the present invention.

In addition, the ability for a heat shock protein or fragment thereof to interact with CD40 and induce cytokine production by the cell expressing CD40 is another useful aspect of the invention.

In another aspect of the invention, the interaction between a mammalian heat shock protein and CD40 may be advantageously exploited to identify new mimetics of mammalian heat shock proteins which have numerous uses, including having immunostimulatory or immunosuppressive properties, as well as new CD40-targeting agents useful for directing antigens and other biomolecules to cells expressing or modified to express CD40, as described in detail hereinabove. Such mimetics are preferably small organic molecules or peptides, and preferably those used in conjugates with another molecule to, for example, induce an immune response thereto, are other than peptide fragments of native heat shock proteins, but the invention is not so limited. Screening methods utilizing the heat shock protein—CD40 interaction may be used in any form or format to identify compounds or agents that promote or inhibit the interaction, for use in identifying compounds, preferably small-molecule compounds but not being so limited, that would be useful as, for example, targeting agents that when conjugated to an antigen or immunogen promote binding to CD40 and uptake, or induce CD40-mediated signaling, the foregoing to promote the induction of an immune response, or, in contrast, inhibit uptake of heat shock protein and any antigen bound thereto by cells expressing CD40, or abrogate CD40 signaling, for the purpose of down-regulating an immune response. The invention embraces the aforementioned screening methods as well as compounds with one or more of the activities described above. The heat shock protein mimetics preferably are not native heat shock protein molecules or fragments of native heat shock proteins.

Such screening methods may employ the fill-length CD40 molecule and mammalian heat shock protein molecules, or interacting fragments of one or both, or cells expressing or made to express CD40 or the exoplasmic region of CD40. In the case of CD40, screening may employ the exoplasmic domain of the molecule (about amino acid 20 to about amino acid 212 of CD40; SEQ ID NO: 318) alone or as a fusion polypeptide, for example, with glutathione S-transferase (GST). Human CD40 is preferred. The hsp70 may be utilized alone in screening, or the N-terminal or ATP-binding domain may be used, such as a polypeptide having from about amino acid 1-5 extending to about amino acid 381 of hsp70, or a polypeptide having from amino acid 5 to amino acid 381 of hsp70 (SEQ ID NO: 1). As described above, a mammalian hsp70, preferably a human hsp70, and most preferably human hsp70, and corresponding fragments thereof, are used. As mentioned above, the format of the screening assay will guide the selection of suitable binding partners.

As mentioned above, the present invention is also directed to modified heat shock protein molecules, such as mammalian hsp70, with modifications to one or more amino acids that results in an increased affinity for binding to CD40. Such modifications may be obtained after determining the interacting sites between mammalian hsp70 and CD40, and particularly between the N-terminal or ATP-binding domain of mammalian hsp70 and the exoplasmic domain of CD40. Further to the discussion above regarding modifications to one or more amino acids in the N-domain of a heat shock protein or a fragment of the N-domain which binds to CD40, as the present inventors have found that the isolated N-domain of hsp70 has greater affinity for CD40 than the full molecule, when neither has a bound peptide, and thus a further aspect of the invention is directed to modified heat shock proteins or muteins thereof with enhanced CD40 binding that have modifications in the C-terminal, or peptide-binding domain (from about amino acid 382 to about amino acid 641 of human hsp70). As the results mentioned above indicate that the presence of the C-domain on intact hsp70 and in particular the C-domain on the intact hsp70 without a loaded peptide appears to interfere with binding to CD40, and in accordance with theory to which the inventors are not bound thereto, the inhibitory influence of the C-domain of full-length hsp70 may be reduced by modifying one or more amino acids therein. Such heat shock protein C-domain muteins may thus have a modification in the C-domain and exhibit increased affinity for CD40. In the instance where the C-domain muteins retain peptide binding activity, such molecules may be employed for antigen delivery as described above. In a preferred embodiment, a heat shock protein mutein is provided with one or more alterations in the C domain which both increases CD40 binding and increases peptide binding. Alternately, the heat shock protein mutein may have at least one N-terminal modification to increase CD40 binding, and at least one C-terminal modification provided to also increase CD40 binding but by reducing the inhibitory activity of the C-domain. A preferred C-domain mutein is expressible recombinantly; other molecules may be expressed and chemically modified thereafter. The invention embraces polynucleotides including degenerate polynucleotides that encode such muteins.

In a further embodiment, the mutein may have at least one modified site in the N-domain to enhance CD40 binding, and at least one modified site in the C-domain to enhance peptide binding and/or to reduce inhibition of N-domain binding to CD40.

As noted above, the invention is also directed to screening methods for compounds that modulate the interaction between a heat shock protein and CD40, for the purpose of identifying compounds including small molecules and peptides, for example, useful as immunostimulatory or immunosuppressive agents by modulating CD40 activity. Such screening methods may be carried out by modulating the interaction between CD40 or a heat shock protein-interacting fragment thereof and a heat shock protein or a CD40-interacting fragment thereof, and may be performed at the in vitro or cellular levels, and may be based on protein-protein interactions, binding, induction of signaling, and other activities described hereinabove and known in the art as measures of the modulation of the interaction of species and the downstream biological effects in cells thereof, including but not limited to steps along the signal transduction pathway. Facile readouts of such assays or screens are be readily devisable by one of skill in the art to enable high-throughput screening to identify candidate compounds that promote or inhibit the aforementioned interactions.

Thus, in any of the protocols and methods herein throughout that employ a heat shock protein or CD40-binding fragment thereof, either alone or in a conjugate with at least another molecule, for any of the various purposes described, the heat shock protein or CD40-binding fragment thereof may be substituted with a peptide, small molecule or other molecule identified as a CD40-binding molecule or hsp mimetic as described above as having the property of modulating the interaction CD40 and hsp70 or the interacting potions thereof.

Prior provisional applications No. 60/342,570 (filed Dec. 26, 2001); No. 60/343,884 (filed Dec. 28, 2001); No. 60/372,620 (filed Apr. 12, 2002), No. 60/399,342 (filed Jul. 29, 2002), and No. 60/414, 834 (filed Sep. 28, 2002) are incorporated herein by reference in their entireties, and the present application claims priority thereto under 35 USC § 119 and §120. All other articles or other citations herein are as well incorporated by reference herein in their entireties.

These and other aspects of the invention will be appreciated by the following description of the figures and the ensuing detailed description of the invention.

### BRIEF DESCRIPTION OF THE FIGURES

FIG. 1 A-B show that lipopolysaccharide (LPS) treatment of ANA1-cells both stimulates binding of Hsp70 and induces expression of CD40.

FIG. 2 A-D show that transfection with human CD40-cDNA renders COS-7 cells active in binding human Hsp70.

FIG. 3 A-C show that binding of Hsp70 to CD40 is direct and depends on ADP.

FIG. 4 A-D show that hsp70 binding to CD40 is mediated by the N-terminal ATPase domain and is competed by Hip.

FIG. 5 A-B show that the presence of a peptide substrate stimulates Hsp70 binding to CD40.

FIG. 6 A-B show that binding of Hsp70 complex to CD40-expressing HEK293T cells induces signaling via p38 and causes peptide uptake.

FIG. 7 depicts a model for the release of Hsp70-peptide complex from a necrotic tumor cell, followed by binding and uptake by an antigen presenting cell.

FIG. 8 shows the pIRES2-EGFP vector encoding full-length CD40.

### DETAILED DESCRIPTION OF THE INVENTION

The present inventors herein unexpectedly and surprisingly discovered the specific binding of mammalian heat shock proteins, and in particular human heat shock proteins hsp70 and hsc70 (generally referred to herein as hsp70 or hsp70 family members), to the cell surface receptor protein molecule CD40. Signaling mediated by hsp70 binding, and uptake of bound hsp70, was also identified. Binding of hsp70 to the exoplasmic domain of CD40 was significantly enhanced when peptide is bound to the hsp70 peptide-binding pocket, which is located in the C-terminal domain of the hsp70 molecule, and binding of hsp70 to CD40 was found to be mediated through the N-terminal (ATP-binding or ATPase) domain of the hsp70 molecule, and not by the peptide-binding, C-terminal domain of the molecule. In fact, the isolated N-terminal domain of hsp70 alone was found to be able to bind tightly to the exoplasmic domain of CD40. Thus, Applicants' findings enable the exploitation of the mammalian heat shock protein-binding property of CD40 in facilitating the cellular uptake of peptides and other molecules associated with the CD40-binding fragments or region of hsp70. As the domain of hsp70 that binds to CD40 is not the domain that binds peptide, both binding to CD40 and delivery of peptides naturally bound to a mammalian hsp70 occurs, in contrast to mycobacterial hsp70 (DnaK), which binds to CD40 by its peptide-binding (C-terminal) domain (Wang et al., 2001, Immunity 15:971-983), and whose binding to CD40 is inhibited when the mycobacterial heat shock protein has a peptide bound in its peptide-binding pocket.

Furthermore, the finding herein that a mammalian hsp70 molecule can both carry a peptide bound to the C-terminal (peptide-binding domain) into a CD40-expressing cell, and also initiate a signal transduction pathway in the CD40-expressing cell that leads to NF-kappaB activation, indicates that the mammalian heat shock protein can carry out its immunological functions in inducing a specific immune response to its carried antigen in the absence of T cell help.

Thus, certain aspects of the present invention are directed to modulation of cellular CD40 levels in vitro, ex vivo and in vivo to modulate an immune response (or any other biological response) to a molecule delivered by covalent attachment or non-covalent association with a native or modified mammalian heat shock protein molecule or a native or modified fragment thereof that binds to CD40. It is also directed to means for identifying small molecule mimetics of mammalian heat shock proteins and modulators of mammalian heat shock protein-mediated immunity by modulating the interaction between a mammalian heat shock protein and CD40. Moreover, the present invention provides a means for identifying and preparing modified heat shock protein molecules and their fragments with enhanced abilities to bind CD40, for enhancing antigen delivery to CD40-expressing cells. In contrast, the invention is also directed to decreasing the immune response mediated by heat shock proteins in instances where this is desirable, particularly in overt or covert autoimmune disease, and adverse exposure to potentially immunogenic toxins, such as allergens and transplant antigens, among others. The binding of heat shock protein to CD40 may be exploited to identify compounds that block this activity and thus may be used to abrogate heat shock protein-mediated adverse immunological events. Such methods and agents may be applied prophylactically, to prevent disease, or therapeutically, to treat disease.

While exploiting the interaction between mammalian heat shock protein and CD40 for immunological purposes is a preferred use for the methods and agents of the present invention, other aspects will be apparent from the description herein. For example, peptides or other pre-selected molecules desired for cellular delivery and mediated through CD40 may be associated with the CD40-binding domain or fragment of hsp70, by covalent or non-covalent means. Modulation of CD40 expression is also an aspect of the invention which may be used to modulate delivery.

CD40 is a transmembrane glycoprotein expressed in various cell types, including but not limited to dendritic cells, B lymphocytes, certain epithelial cells, among a variety of non-lymphocytic cell types. It is expressed on other cells during pathological states. It is a member of the nerve growth factor receptor/tumor necrosis factor receptor family. The properties of CD40 as a signal-transduction molecule have been established in numerous studies. A natural ligand for CD40 has been identified, termed CD40 ligand or CD40L (also known as CD154), and the interaction between various immune cells, including phagocytes such as dendritic cells and other antigen-presenting cells, and T cells and B cells, through CD40L and CD40, plays an important role in the immune response. Such binding, through altering multimerization of CD40 by binding to its ligand, mediates expression of adhesion molecules, cytokines, matrix-degrading enzymes, prothrombotic activities and apoptotic mediators, among other effects. As such, CD40 signaling has been associated with pathogenic processes including chronic inflammatory disease, autoimmune diseases, neurodegenerative disorders, graft-versus-host disease, cancer and atherosclerosis. See, for example, Diehl et al., J. Mol. Med. 2000;78(7):363-71 and van Kooten and Banchereau, J. Leukoc. Biol. 2000 Jan;67(1):2-17, and articles cited therein, for reviews on CD40). It is toward modulating the signaling resulting from the interaction between CD40 and heat shock proteins that an aspect of the present invention is directed.

The present invention is thus in one aspect broadly directed to modulating the binding of heat shock proteins and preferably a complex of a heat shock protein and any molecule associated therewith by various cell types, by altering the expression or binding activity of CD40 on such cells. In one embodiment, the mammalian heat shock protein is human hsp70. Events attendant to binding of the heat shock protein-molecular complex to CD40, such as signaling and internalization, are also modulatable by the compositions and methods of the present invention and are useful for inducing changes in cellular physiology as well as facilitating delivery of certain molecules such as antigenic molecules and epitopes, into cells expressing, induced to express, or induced to increase expression of CD40. Such delivery may be for the purpose of altering the genotype and possibly the phenotype of the cell, or, preferably, to enhance immunity in a mammal to an antigenic molecule bound to or associated with the heat shock protein. In a further aspect of the invention, the signaling properties of CD40 induced by binding of and thus attributable to heat shock proteins may be advantageously exploited in protocols and methods in which CD40-mediated signaling is desired. While the invention is preferably directed to mammalian heat shock proteins, more preferably human and most preferably human hsp70, it is not so limiting.

The present invention is also directed to new delivery molecules that deliver antigens or other preselected molecules to cells expressing CD40 by taking advantage of the affinity between CD40 and heat shock protein, and in particular mammalian heat shock protein, and more particularly the N-domain (ATP-binding or ATPase domain) of a mammalian heat shock protein, preferably human hsp70, as discovered by the inventors herein. Such new delivery molecules include fragments of heat shock proteins that bind CD40 and thus can carry a preselected molecule such as a MHC/HLA Class I peptide, by either non-covalently binding to the peptide-binding pocket of the heat shock protein, or by covalently attaching the preselected molecule to the CD40-binding heat shock protein fragment. Such new delivery molecules also include modifications of full length heat shock proteins or fragments thereof, such modifications increasing the affinity of the heat shock protein or fragment for CD40, optionally including increasing the affinity of the heat shock protein or fragment for a peptide to be loaded/carried.

This, the fragment of a mammalian heat shock protein molecule, preferably a mammalian hsp70 family member, more preferably a human hsp70 family member and most preferably human hsp70, that is useful for the purposes herein is not the C-terminal (peptide-binding) domain or a fragment thereof, but the N-terminal or ATPase (ATP binding) domain and fragments thereof, the N-domain which does not bind peptide. In particular, the fragment of hsp70 is all of part of the N-domain, which extends from amino acid 1 to about amino acid 381 of the hsp70 molecule. The invention thus is also directed to conjugates and fusion polypeptides comprising the aforementioned N-domain of a heat shock protein, preferably a mammalian hsp70 and more preferably human hsp70, or CD40-binding fragments thereof, and another molecule, preferably a protein or peptide, more preferably at least one antigen or epitope, and most preferably at least an epitope of a cancer or infectious disease. The compositions may comprise at least one CD40-binding domain and at least one immunogenic or antigenic domains, but there may be more than one of either. In a preferred embodiment, the composition of the invention comprises a single CD40-binding domain and multiple antigens or epitopes, preferably co-linearly expressed, such that the composition may elicit an effective immune response to a target disease. As will be known to the skilled artisan, a number of different antigens or epitopes may be required to effectively treat or prevent a particular disease, especially when there is antigenic variation among the causative organism, or variations in cellular expression patterns of target self-antigens in diseased cells, and moreover, the HLA haplotype of an individual dictates the particular effective epitope(s) for the immunogen. For treatment of a variety of HLA-type individuals, a composition of the invention will have a plurality of epitopes that are disease specific, and may have a further plurality that are HLA specific, depending on the spectrum of patients desirably treated with a single immunogenic composition of the invention. The invention also embraces polynucleotides encoding such conjugates, including degenerate sequences thereof, and recombinant expression of such polynucleotides. Such polynucleotides may be used for recombinant expression of the compositions in vitro for manufacturing purposes, or used therapeutically as vaccines for the prophylaxis or treatment of the epitope(s)-related disease. Thus, DNA vaccines based on the compositions of the invention are fully embraced herein.

The word “fragment” as used herein refers to a portion of a parent molecule that may have as little as one amino acid missing in comparison to the parent molecule, or may be a smaller fragment to about at least 6 amino acids, but it is not so limiting.

In contrast to the above, modulation of CD40 expression to achieve a decreased binding, signaling or internalization by cells of or by heat shock proteins, particularly endogenous heat shock proteins, and peptides bound thereto, is another aspect of the invention. Such modulation may usefully interfere with induction or recurrence of an undesirable immune response to an endogenous or environmental antigen, useful, among other purposes, for the treatment of autoimmune diseases, immune responses to transplant antigens, and the like.

Tumor and viral antigens elicit potent immune responses by heat-shock protein-dependent uptake by antigen presenting cells of antigenic peptides with subsequent presentation by MHC (murine) Class I molecules (HLA Class I in humans). Receptors on antigen presenting cells that specifically bind, signal and internalize mammalian heat shock protein-peptide complexes had not previously been identified. The present inventors have shown that cells expressing CD40 specifically bind and internalize human hsp70 with bound peptide. Binding of hsp70-peptide complex to the exoplasmic domain of CD40 is mediated by the N-terminal nucleotide binding domain of hsp70 in its ADP-state. The hsp70 cochaperone Hip, but not the bacterial hsp70 homologue DnaK, competes for formation of the hsp70-CD40 complex. As noted above, binding of hsp70-ADP to CD40 is strongly increased in the presence of hsp70 peptide substrate, and furthermore, induces signaling via p38. These studies indicate that CD40 is a co-chaperone-like receptor mediating the uptake of exogenous hsp70-peptide complexes by macrophages and dendritic cells, by way of example.

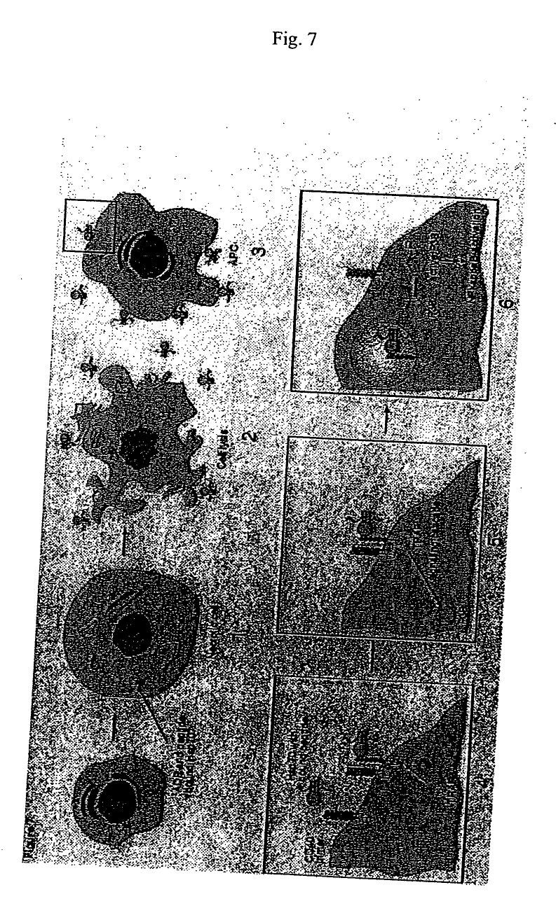

These results establish the cell surface protein CD40 as a receptor for exogenously-added human hsp70-peptide complexes. The functional properties of the CD40-hsp70 interaction ensure surface binding and uptake of hsp70-associated peptide. While Applicants are not required to disclose the mechanism by which their invention operates, and further are not bound by any such disclosure, FIG. 7 describes a model for the binding of peptide antigen to Hsp70 in a tumor cell, followed by necrotic cell lysis and CD40-mediated uptake of the Hsp70-peptide complex by an antigen presenting cell (APC). Peptide binding to Hsp70 would be facilitated by the high intracellular concentration of ATP and the activity of the Hsp70 cochaperone Hsp40 in catalyzing peptide loading (Minami et al., 1996, J. Biol. Chem. 271:19617-19624). During cell necrosis the internal concentration of ATP relative to ADP drops markedly (Bradbury et al., 2000, J. Immunol. Methods 240:79-92). A further dilution of ATP (and of Hsp70 cochaperones) would occur upon lysis and release of cytosol content into the extracellular medium. As a result, peptide-bound Hsp70 remains in its ADP-state, the stability of which determines the half-life of the Hsp70-peptide complex. Importantly, although peptide loading onto Hsp70 is possible in the absence of nucleotide with low efficiency (Minami et al., 1996, idem), low nucleotide concentration would prohibit the re-formation of an Hsp70-ADP-peptide complex in the extracellular space. Thus, the strong preference of CD40 for Hsp70-ADP-peptide ensures not only the binding of peptide-loaded Hsp70, but would also guarantee that intracellular peptide antigen is made available for cross priming. Thus, the uptake of circulating extracellular peptides, potentially triggering autoimmune reactions, would be avoided. Applicants are not bound by the foregoing theory.

Binding of heat shock protein to CD40 is mediated by the ATPase domain of hsp70 and is enhanced in the ADP-loaded state of the chaperone, which binds peptide tightly. Moreover, complex formation between CD40 and hsp70 is strongly enhanced by the presence of hsp70 peptide substrate. Thus, the CD40-hsp70 interaction shares important functional features with the interaction between hsp70 and certain intracellular cochaperones, such as Hip. As noted above, hsp70 binding to CD40 is enhanced by the interaction of substrate peptide bound to the C-terminal domain of hsp70. The studies described herein indicate that CD40 interacts with the ATPase domain of Hsp70, in the presence of ADP.

As mentioned above, the functional features of the CD40-Hsp70 interaction may be adapted to a role in the uptake of Hsp70-peptide complexes into APCs for cross priming. The various embodiments of the invention directed to enhancing the immune response are thusly directed.

While various aspects of the present invention are directed to mammalian heat shock proteins, preferably to the hsp70 family of heat shock proteins, more preferably to human and most preferably human hsp70, it is not so limiting. Other heat shock proteins capable of binding the CD40 include other members of the hsp70 family, such as hsc70, BiP, hsp110, hsp72 and hsp73, as well as other heat shock proteins, such as but not limited to hsp40, hsp60, hsp90, gp96, calreticulin, grp170, PDI, hsp100, smhsp, and hsp27. A discussion on members of the hsp70 family as well as other heat shock proteins in general may be found in Rothman, 1989, Cell 59:591-601 and Pelham, 1986, Cell 49:959-961, and articles cited therein. The skilled artisan can readily identify CD40-binding activity in native heat shock proteins or fragments thereof.

In a preferred but non-limiting embodiment of all of the utilities of heat shock proteins, and in particular hsp70, herein, the term heat shock protein embraces fragments thereof comprising the CD40-binding portion of any of the foregoing exemplary heat shock proteins, or fusion polypeptides or other molecules at least comprising a CD40-binding portion of a heat shock protein. The heat shock protein or the CD40-binding fragment may be in a covalent or non-covalent complex with a pre-selected biomolecule, such as those with affinity for non-covalent binding to a heat shock protein or fragment thereof, those that can be made to have an affinity for binding to a heat shock protein, and those that are covalently bound to the heat shock protein or fragment thereof. As mentioned above, the biomolecule or antigen may be isolated from diseased cells as a complex with heat shock proteins present therein, as natural complexes, or they may be prepared in vitro. In a preferred embodiment, the heat shock protein is non-covalently bound to a hybrid antigen, the hybrid antigen comprising the pre-selected molecule, which may be, for example, an antigenic or immunogenic domain, and a heat-shock-protein-binding domain. ADP is present in such formulations. Non-limiting examples of selections of such heat shock protein binding domains are described in Moroi et al., 2000, Proc. Nat. Acad. Sci. U.S.A. 97:3485-3490; PCT/US96/13363 (WO9706821); PCT/US98/22335 (WO9922761); PCT/US01/12567 (WO0179259); PCT/US01/12568 (WO0178772); and PCT/US01/12449 (WO0178655), and citations therein, in particular Gragerov et al., J. Molec. Biol. 235:848-854 (1994), Flynn et al., Science 245: 385-390 (1989), Auger et al. Nature Medicine 2:306-310 (1996), Blond-Elguindi et al., Cell 75:717-728 (1993), among others. Other covalent and non-covalent complexes of heat shock proteins and antigens or epitopes isolated from cells or prepared in vitro are described in U.S. Pat. Nos. 5,997,873; 6,168,793; 5,710,119; 5,961,979; 6,048,530; 5,935,576; 6,030,618; 5,837,251; 6,017,544; 6,335,183; 6,338,952; 6,410,028; 6,447,781; PCT/US96/14556 (WO9710000), PCT/CA98/00246 (WO9907860), PCT/CA97/00897 (WO9823735); and PCT/US02/03460 (WO0262959); all of which are incorporated herein by reference in their entireties. The aforementioned complexes comprising a mammalian and more preferably a human heat shock protein are preferred. Complexes comprising human hsp70 are most preferred.

By way of non-limiting example, the following heat shock protein binding domain peptides (depicted by one-letter code) may be linked to a pre-selected molecule for enhancing the binding of the pre-selected molecule to a mammalian heat shock protein, preferably a member of the hsp70 family and most preferably hsp70, for the various uses described herein.

Hy (W/Xaa) HyXaaHyXaaHy (SEQ ID NO: 2), where each Hy is independently a hydrophobic amino acid such as but not limited to tryptophan, leucine and phenylalanine, and Xaa is any amino acid; HWDFAWPW (SEQ ID NO: 3); NLLRLTGW (SEQ ID NO: 4); FYQLALTW (SEQ ID NO: 5); RKLFFNLRW (SEQ ID NO: 6); QKRAA (SEQ ID NO: 7); RRRAA (SEQ ID NO: 8); LFWPFEWI (SEQ ID NO: 9); FTYGSRWL (SEQ ID NO: 10); FWGLWPWE (SEQ ID NO: 11); DGVGSFIG (SEQ ID NO: 12); KRQIYTDLEMNRLGK (SEQ ID NO: 13); PLSQETFSGLWKLLPPEDG (SEQ ID NO: 14); YVDRFIGW (SEQ ID NO: 15); VGIDLGTTYSC (SEQ ID NO: 16); THCDGFQNE (SEQ ID NO: 17); EGMIDGWYGFRHQNC (SEQ ID NO: 18); CIRCQLSGNS (SEQ ID NO: 19); SQKVPTSQIKC (SEQ ID NO: 20); KGLRNMELDTYIQRK (SEQ ID NO: 21); SKYIPRRKPRFLSSL (SEQ ID NO: 22); KPRFLSSLVGILK (SEQ ID NO: 23); RFHAMGVDSKYIPRR (SEQ ID NO: 24); GKWVYI (SEQ ID NO: 25); AKRETK (SEQ ID NO: 26); KWVHLF (SEQ ID NO: 27); RLVLVL (SEQ ID NO: 28); WKWGIY (SEQ ID NO: 29); SSHASA (SEQ ID NO: 30); WGPWSF (SEQ ID NO: 31); AIPGKV (SEQ ID NO: 32); RVHDPA (SEQ ID NO: 33); RSVSSF (SEQ ID NO: 34); LGTRKG (SEQ ID NO: 35); KDPLFN (SEQ ID NO: 36); LSQHTN (SEQ ID NO: 37); NRLLLT (SEQ ID NO: 38); YPLWVI (SEQ ID NO: 39); LLIIDR (SEQ ID NO: 40); RVISLQ (SEQ ID NO: 41); EVSRED (SEQ ID NO: 42); SILRST (SEQ ID NO: 43); PGLVWL (SEQ ID NO: 44); VKKLYI (SEQ ID NO: 45); NNRLLD (SEQ ID NO: 46); SKGRWG (SEQ ID NO: 47); IRPSGI (SEQ ID NO: 48); ASLCPT (SEQ ID NO: 49); DVPGLR (SEQ ID NO: 50); RHREVQ (SEQ ID NO: 51); LARKRS (SEQ ID NO: 52); SVLDHV (SEQ ID NO: 53); NLLRRA (SEQ ID NO: 54); SGISAW (SEQ ID NO: 55); FYPWVR (SEQ ID NO: 56); KLPXaaLPL (SEQ ID NO: 57); TPTLSD (SEQ ID NO: 58); THSLIL (SEQ ID NO: 59); LLLLSR (SEQ ID NO: 60); LLRVRS (SEQ ID NO: 61); ERRSRG (SEQ ID NO: 62); RMLQLA (SEQ ID NO: 63); RGWANS (SEQ ID NO: 64); RPFYSY (SEQ ID NO: 65); SSSWNA (SEQ ID NO: 66); LGHLEE (SEQ ID NO: 67); SAVTNT (SEQ ID NO: 68); KWVHLFG (SEQ ID NO: 69); NRLLLTG (SEQ ID NO: 70); LRRWSLG (SEQ ID NO: 71); ARLLLTG (SEQ ID NO: 72); NALLLTG (SEQ ID NO: 73); NRLALTG (SEQ ID NO: 74); NLLRLTG (SEQ ID NO: 75); NRLWLTG (SEQ ID NO: 76); NRLLLAG (SEQ ID NO: 77); MQERITLKDYAM (SEQ ID NO: 78); NDLLLTG (SEQ ID NO: 79); RGYVYQGL (SEQ ID NO: 80); KFERQ (SEQ ID NO: 81); HTTVYGAG (SEQ ID NO: 82); TETPYPTG (SEQ ID NO: 83); LTTPFSSG (SEQ ID NO: 84); GVPLTMDG (SEQ ID NO: 85); KLPTVLRG (SEQ ID NO: 86); CRFHGNRG (SEQ ID NO: 87); YTRDFEAG (SEQ ID NO: 88); SSAAGPRG (SEQ ID NO: 89); SLIQYSRG (SEQ ID NO: 90); DALMWP Xaa G (SEQ ID NO: 91); SS Xaa SLYIG (SEQ ID NO: 92); FNTSTRTG (SEQ ID NO: 93); TVQHVAFG (SEQ ID NO: 94); DYSFPPLG (SEQ ID NO: 95); VGSMESLG (SEQ ID NO: 96); F Xaa PMI Xaa SG (SEQ ID NO: 97); APPRVTMG (SEQ ID NO: 98); IATKTPKG (SEQ ID NO: 99); KPPLFQIG (SEQ ID NO: 100); YHTAHNMG (SEQ ID NO: 101); SYIQATHG (SEQ ID NO: 102); SSFATFLG (SEQ ID NO: 103); TTPPNFAG (SEQ ID NO: 104); ISLDPRMG (SEQ ID NO: 105); SLPLFGAG (SEQ ID NO: 106); NLLKTTLG (SEQ ID NO: 107); DQNLPRRG (SEQ ID NO: 108); SHFEQLLG (SEQ ID NO: 109); TPQLHHGG (SEQ ID NO: 110); APLDRITG (SEQ ID NO: 111); FAPLIAHG (SEQ ID NO: 112); SWIQTFMG (SEQ ID NO: 113); NTWPHMYG (SEQ ID NO: 114); EPLPTTLG (SEQ ID NO: 115); HGPHLFNG (SEQ ID NO: 116); YLNSTLAG (SEQ ID NO: 117); HLHSPSGG (SEQ ID NO: 118); TLPHRLNG (SEQ ID NO: 119); SSPREVHG (SEQ ID NO: 120); NQVDTARG (SEQ ID NO: 121); YPTPLLTG (SEQ ID NO: 122); HPAAFPWG (SEQ ID NO: 123); LLPHSSAG (SEQ ID NO: 124); LETYTASG (SEQ ID NO: 125); KYVPLPPG (SEQ ID NO: 126); APLALHAG (SEQ ID NO: 127); YESLLTKG (SEQ ID NO: 128); SHAASGTG (SEQ ID NO: 129); GLATVKSG (SEQ ID NO: 130); GATSFGLG (SEQ ID NO: 131); KPPGPVSG (SEQ ID NO: 132); TLYVSGNG (SEQ ID NO: 133); HAPFKSQG (SEQ ID NO: 134); VAFTRLPG (SEQ ID NO: 135); LPTRTPAG (SEQ ID NO: 136); ASFDLLIG (SEQ ID NO: 137); RMNTEPPG (SEQ ID NO: 138); KMTPLTTG (SEQ ID NO: 139); ANATPLLG (SEQ ID NO: 140); TIWPPPVG (SEQ ID NO: 141); QTKVMTTG (SEQ ID NO: 142); NHAVFASG (SEQ ID NO: 143); LHAAXaa TSG (SEQ ID NO: 144); TWQPYFHG (SEQ ID NO: 145); APLALHAG (SEQ ID NO: 146); TAHDLTVG (SEQ ID NO: 147); NMTNMLTG (SEQ ID NO: 148); GSGLSQDG (SEQ ID NO: 149); TPIKTIYG (SEQ ID NO: 150); SHLYRSSG (SEQ ID NO: 151); YTLVQPL (SEQ ID NO: 152); TPDITPK (SEQ ID NO: 153); TYPDLRY (SEQ ID NO: 154); DRTHATS (SEQ ID NO: 155); MSTTFYS (SEQ ID NO: 156); YQHAVQT (SEQ ID NO: 157); FPFSAST (SEQ ID NO: 158); SSFPPLD (SEQ ID NO: 159); MAPSPPH (SEQ ID NO: 160); SSFPDLL (SEQ ID NO: 161); HSYNRLP (SEQ ID NO: 162); HLTHSQR (SEQ ID NO: 163); QAAQSRS (SEQ ID NO: 164); FATHHIG (SEQ ID NO: 165); SMPEPLI (SEQ ID NO: 166); IPRYHLI (SEQ ID NO: 167); SAPHMTS (SEQ ID NO: 168); KAPVWAS (SEQ ID NO: 169); LPHWLLI (SEQ ID NO: 170); ASAGYQI (SEQ ID NO: 171); VTPKTGS (SEQ ID NO: 172); EHPMPVL (SEQ ID NO: 173); VSSFVTS (SEQ ID NO: 174); STHFTWP (SEQ ID NO: 175); GQWWSPD (SEQ ID NO: 176); GPPHQDS (SEQ ID NO: 177); NTLPSTI (SEQ ID NO: 178); HQPSRWV (SEQ ID NO: 179); YGNPLQP (SEQ ID NO: 180); FHWWWQP (SEQ ID NO: 181); ITLKYPL (SEQ ID NO: 182); FHWPWLF (SEQ ID NO: 183); TAQDSTG (SEQ ID NO: 184); FHWWWQP (SEQ ID NO: 185); FHWWDWW (SEQ ID NO: 186); EPFFRMQ (SEQ ID NO: 187); TWWLNYR (SEQ ID NO: 188); FHWWWQP (SEQ ID NO: 189); QPSHLRW (SEQ ID NO: 190); SPASPVY (SEQ ID NO: 191); FHWWWQP (SEQ ID NO: 192); HPSNQAS (SEQ ID NO: 193); NSAPRPV (SEQ ID NO: 194); QLWSIYP (SEQ ID NO: 195); SWPFFDL (SEQ ID NO: 196); DTTLPLH (SEQ ID NO: 197); WHWQMLW (SEQ ID NO: 198); DSFRTPV (SEQ ID NO: 199); TSPLSLL (SEQ ID NO: 200); AYNYVSD (SEQ ID NO: 201); RPLHDPM (SEQ ID NO: 202); WPSTTLF (SEQ ID NO: 203); ATLEPVR (SEQ ID NO: 204); SMTVLRP (SEQ ID NO: 205); QIGAPSW (SEQ ID NO: 206); APDLYVP (SEQ ID NO: 207); RMPPLLP (SEQ ID NO: 208); AKATPEH (SEQ ID NO: 209); TPPLRIN (SEQ ID NO: 210); LPIHAPH (SEQ ID NO: 211); DLNAYTH (SEQ ID NO: 212); VTLPNFH (SEQ ID NO: 213); NSRLPTL (SEQ ID NO: 214); YPHPSRS (SEQ ID NO: 215); GTAHFMY (SEQ ID NO: 216); YSLLPTR (SEQ ID NO: 217); LPRRTLL (SEQ ID NO: 218); TSTLLWK (SEQ ID NO: 219); TSDMKPH (SEQ ID NO: 220); TSSYLAL (SEQ ID NO: 221); NLYGPHD (SEQ ID NO: 222); LETYTAS (SEQ ID NO: 223); AYKSLTQ (SEQ ID NO: 224); STSVYSS (SEQ ID NO: 225); EGPLRSP (SEQ ID NO: 226); TTYHALG (SEQ ID NO: 227); VSIGHPS (SEQ ID NO: 228); THSHRPS (SEQ ID NO: 229); ITNPLTT (SEQ ID NO: 230); SIQAHHS (SEQ ID NO: 231); LNWPRVL (SEQ ID NO: 232); YYYAPPP (SEQ ID NO: 233); SLWTRLP (SEQ ID NO: 234); NVYHSSL (SEQ ID NO: 235); NSPHPPT (SEQ ID NO: 236); VPAKPRH (SEQ ID NO: 237); HNLHPNR (SEQ ID NO: 238); YTTHRWL (SEQ ID NO: 239); AVTAAIV (SEQ ID NO: 240); TLMHDRV (SEQ ID NO: 241); TPLKVPY (SEQ ID NO: 242); FTNQQYH (SEQ ID NO: 243); SHVPSMA (SEQ ID NO: 244); HTTVYGA (SEQ ID NO: 245); TETPYPT (SEQ ID NO: 246); LTTPFSS (SEQ ID NO: 247); GVPLTMD (SEQ ID NO: 248); KLPTVLR (SEQ ID NO: 249); CRFHGNR (SEQ ID NO: 250); YTRDFEA (SEQ ID NO: 251); SSAAGPR (SEQ ID NO: 252); SLIQYSR (SEQ ID NO: 253); DALMWP Xaa (SEQ ID NO: 254); SS Xaa SLYI (SEQ ID NO: 255); FNTSTRT (SEQ ID NO: 256); TVQHVAF (SEQ ID NO: 257); DYSFPPL (SEQ ID NO: 258); VGSMESL (SEQ ID NO: 259); F Xaa PMI Xaa S (SEQ ID NO: 260); APPRVTM (SEQ ID NO: 261); IATKTPK (SEQ ID NO: 262); KPPLFQI (SEQ ID NO: 263); YHTAHNM (SEQ ID NO: 264); SYIQATH (SEQ ID NO: 265); SSFATFL (SEQ ID NO: 266); TTPPNFA (SEQ ID NO: 267); ISLDPRM (SEQ ID NO: 268); SLPLFGA (SEQ ID NO: 269); NLLKTTL (SEQ ID NO: 270); DQNLPRR (SEQ ID NO: 271); SHFEQLL (SEQ ID NO: 272); TPQLHHG (SEQ ID NO: 273); APLDRIT (SEQ ID NO: 274); FAPLIAH (SEQ ID NO: 275); SWIQTFM (SEQ ID NO: 276); NTWPHMY (SEQ ID NO: 277); EPLPTTL (SEQ ID NO: 278); HGPHLFN (SEQ ID NO: 279); YLNSTLA (SEQ ID NO: 280); HLHSPSG (SEQ ID NO: 281); TLPHRLN (SEQ ID NO: 282); SSPREVH (SEQ ID NO: 283); NQVDTAR (SEQ ID NO: 284); YPTPLLT (SEQ ID NO: 285); HPAAFPW (SEQ ID NO: 286); LLPHSSA (SEQ ID NO: 287); LETYTAS (SEQ ID NO: 288); KYVPLPP (SEQ ID NO: 289); APLALHA (SEQ ID NO: 290); YESLLTK (SEQ ID NO: 291); SHAASGT (SEQ ID NO: 292); GLATVKS (SEQ ID NO: 293); GATSFGL (SEQ ID NO: 294); KPPGPVS (SEQ ID NO: 295); TLYVSGN (SEQ ID NO: 296); HAPFKSQ (SEQ ID NO: 297); VAFTRLP (SEQ ID NO: 298); LPTRTPA (SEQ ID NO: 299); ASFDLLI (SEQ ID NO: 300); RMNTEPP (SEQ ID NO: 301); KMTPLTT (SEQ ID NO: 302); ANATPLL (SEQ ID NO: 303); TIWPPPV (SEQ ID NO: 304); QTKVMTT (SEQ ID NO: 305); NHAVFAS (SEQ ID NO: 306); LHAA Xaa TS (SEQ ID NO: 307); TWQPYFH (SEQ ID NO: 308); APLALHA (SEQ ID NO: 309); TAHDLTV (SEQ ID NO: 310); NMTNMLT (SEQ ID NO: 311); GSGLSQD (SEQ ID NO: 312); TPIKTIY (SEQ ID NO: 313); SHLYRSS (SEQ ID NO: 314); HGQAWQF (SEQ ID NO: 315); NLLRLTG (SEQ ID NO: 316) and FHWWW (SEQ ID NO: 317).

In another embodiment, the heat shock protein binding domain may be directed to bind to a different part of the mammalian heat shock protein that those aforementioned, and the heat shock protein-binding domains of the invention are not limited to binding to any particular portion of the heat shock protein molecule. In a non-limiting example, the peptide IFAGIKKKAERADLIAYLKQATAK (Greene et al., 1995, J. Biol. Chem. 270:2967-2973; SEQ ID NO: 331) or a heat shock protein-binding fragment of this peptide, is used in any of the conjugates of the invention to facilitate the binding of a pre-selected molecule to a heat shock protein or CD40-binding fragment thereof. The aforementioned peptide and its fragments is particularly useful for binding pre-selected molecules to CD40-binding heat shock protein fragments that are lacking the natural peptide-binding domain or pocket.

In addition to the aforementioned peptides that bind to heat shock proteins, the binding may be achieved through the use of an organic molecule or compound with heat shock protein binding activity. For example, suitable molecules include members of the benzoquinone ansamycin antibiotics, such as herbimycin A, geldanamycin, macmimycin I, mimosamycin, and kuwaitimycin (Omura et al., 1979, J. Antibiotics 32:255-261), or structurally related compounds, and analogs or derivatives thereof. These molecules may be conjugated though established chemical means to the pre-selected molecules described herein throughout, for facilitating the binding of the molecule to a heat shock protein or CD40-binding fragment thereof.

The doses of the CD40 modulating agents and heat shock protein molecules or fragments and their associated molecules for use in the various aspects of the invention herein described may be readily determined in models or patients by one of skill in the art. Immunological assays for induction of a specific immune response are well established and are routinely carried out. Measurements of antigen-specific T cells using a tetramer assay, measurements of intracellular cytokine staining after exposure to antigen, cytokine secretion assays, and the like, may be applied to determining optimal doses of the formulations of CD40 modulating compounds and heat shock protein molecules of the invention. Moreover, formulations of the agents and heat shock proteins and fragments of the invention form mammalian administration may be prepared in pharmaceutically-acceptable excipients, diluents, carriers and the like, according to standard formulating protocols for pharmaceutical products.

The following sections describe generally the various aspects of the invention. While they are set forth under different headings merely for convenience, many of the methods and compositions used thereby are common among the aspects and are interchangeably useful. While the preferred aspect in the various embodiments is to mammalian heat shock proteins, more preferably human heat shock proteins and most preferably human hsp70, it is not so limiting. This will be clear to one of skill in the art.

### Enhancing Heat Shock Protein/antigen Complex Uptake by Increasing CD40 Expression

As noted above, the present inventors have identified CD40 as a mammalian heat-shock-protein-binding molecule (i.e., a cell surface receptor for a mammalian heat shock protein, preferably a human heat shock protein, more preferably mammalian hsp70 family members and most preferably human hsp70). Moreover, CD40 is capable of binding and inducing signaling when a peptide is bound thereto; in fact, it is significantly enhanced when a peptide is bound. In this aspect, CD40 expression may be induced in a cell or increased in a cell, either in vitro, ex vivo, in vivo, or any combination of the foregoing, in order to enhance the uptake of a mammalian heat shock protein or uptake of a fragment, fusion polypeptide, or other molecule comprising a portion of a heat shock protein which binds to CD40 (herein throughout referred to as the CD40-binding portion of a heat shock protein, or syntactic variants thereof). In one aspect, increased CD40 expression enhances the uptake and processing of endogenous (i.e., naturally present) complexes of heat shock proteins and associated antigens, such as peptide antigens, which are bound to the heat shock protein via the peptide binding pocket of the heat shock protein. Preferably the heat shock protein or fragment taken up under such conditions has bound thereto, covalently or non-covalently, an antigenic or immunogenic molecule, for which an immune response thereto is desirably induced or alternatively, tolerized upon uptake. The immune response may be elicitation of effector T cells, induction of a humoral response, of both; or under other conditions, tolerization to or abrogation of a cellular, humoral, or both responses to a particular antigen or antigens. The foregoing are merely exemplary of immunotherapeutic utilities of the invention, and further utilities of the invention involving non-immunotherapeutic uses are fully embraced herein, such as but not limited to enhancing heat-shock-protein-mediated delivery of molecules into cells expressing or induced to express CD40, as will be elaborated upon below. Reduction or inhibition of CD40-mediated heat shock protein delivery is also embraced herein.

By way of example, a sample of phagocytes from a mammalian animal, preferably but not limited to a human patient, such as those in whole blood or isolated from whole blood, may be exposed ex vivo to an agent capable of inducing or increasing CD40 expression. As noted above, such agents include but are not limited to a vector encoding CD40, or an agent such as a calcium ionophore, cytokine, or LPS, as hereinabove described, that on exposure to the phagocytes induces and/or upregulates CD40 expression. At least one agent may be used, but more than one agent may be used, such as initially using an expressible polynucleotide encoding CD40, and subsequently, an agent that increases expression of the polynucleotide. A CD40 construct comprising an inducible promoter may be transfected into target cells, then the cells may be exposed to the inducer. Moreover, one agent may be used in one setting, such as ex vivo, and another agent used in vivo: for example, phagocytes isolated from an individual may be induced to express CD40 by introducing a polynucleotide encoding CD40 ex vivo, and after readministration to the individual, a cytokine is administered systemically or locally to further increase CD40 expression. After exposure, the phagocytes may be washed to remove excess agent, and the phagocytes returned to the individual. In another embodiment, allogeneic phagocytes, such as from a cell culture, donor individual, or other source, are thusly treated in vitro and then administered.

As mentioned above, in all of the embodiments herein, the methods that employ a polynucleotide encoding CD40, the polynucleotide may encode a fragment of or a amino acid sequence-sequence modification of native CD40 wherein expression of the encoding polynucleotide results in the cell exhibiting CD40-like heat shock protein-binding affinity, signaling, or internalization properties, and any combination of the foregoing. In some cases, all three CD40 activities will be required; in others; two of three or just binding may be adequate for the desired outcome of the particular method. A modified CD40 protein with enhanced heat shock protein binding affinity is also embraced within the present invention for the various methods described herein.

Delivery of the aforementioned expressible polynucleotide encoding CD40 or a fragment thereof that renders a cell capable of at least binding a mammalian heat shock protein may be achieved by any number of methods well known in the art. For example, a naked DNA vector (see, e.g., Ulmer et al., 1993, Science 259:1745-1749), a DNA vector transporter (e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed March 15, 1990), or a viral vector containing the desired exp gene can be injected into cells or tissues. Suitable viral vectors include retroviruses that are packaged in cells with amphotropic host range (see Miller, 1990, Human Gene Ther. 1:5-14; Ausubel et al., Current Protocols in Molecular Biology, § 9), and attenuated or defective DNA virus, such as but not limited to herpes simplex virus (HSV) (see, e.g., Kaplitt et al., 1991, Molec. Cell. Neurosci. 2:320-330), papillomavirus, Epstein Barr virus (EBV), adenovirus (see, e.g., Stratford-Perricaudet et al., 1992, J. Clin. Invest. 90:626-630), adeno-associated virus (AAV) (see, e.g., Samulski et al., 1987, J. Virol. 61:3096-3101; Samulski et al., 1989, J. Virol. 63:3822-3828), and the like. Defective viruses, which entirely or almost entirely lack viral genes, are one embodiment. Defective virus is not infective after introduction into a cell.

Vectors containing the polynucleotide of the invention can be introduced into the desired host by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990).

Administration of the foregoing polynucleotide in whichever form is employed may be by any parenteral route, including but not limited to intramuscular, intraperitoneal, intravenous, and the like. Administration directly, or by targeting or choice of a viral vector, indirectly, to lymphoid tissues, e.g., lymph nodes or spleen, is one embodiment.

The amount of agent used to transfect, induce or increase expression of CD40 may be readily determined by one of skill in the art, depending on the agent, the numbers and type of cell or mixture of cells desirably treated, and the conditions of treatment, such as duration of ex-vivo exposure.

In the instance where uptake and processing of endogenous heat shock protein complexes with antigens is desirably increased in phagocytes, such as dendritic cells, the methods of the invention may be applied in vitro, in vivo or ex vivo to phagocytes, in order to increase the expression of CD40 and consequently heat shock protein uptake and processing. Examples of protocols for carrying out such procedures are described in the Summary of the Invention, above, and are merely exemplary and non-limiting.

In addition to enhancing the uptake and processing of endogenous heat shock proteins, protocols involving the aforementioned manipulations plus exposure to exogenous heat shock protein/antigen complexes either in ex-vivo or in-vivo are embraced hereby as well.

In an ex-vivo aspect, a method is provided for enhancing the uptake of a heat shock protein by a cell by exposing the cell ex vivo to an agent capable of increasing CD40 expression by the cell. In this aspect, cells which are identified for taking up an antigen or immunogen in a complex with a heat shock protein for the purpose of presenting the antigen or fragments thereof on MHC/HLA molecules for the subsequent purpose of inducing an immune response is embraced herein. Cells are preferably phagocytes and include professional as well as non-professional phagocytes, such as antigen presenting cells, including but not limited to dendritic cells, macrophages, epithelial cells, endothelial cells, etc. The dendritic cells may be lymphoid derived or myeloid derived. Preferably for the ex-vivo aspect of the invention, antigen presenting cells that are present in or optionally isolated from whole blood (peripheral blood mononuclear cells, or PBMCs) are preferred. Although it is preferred that the antigen presenting cells are isolated from the individual for which the ex-vivo methods of this aspect of the invention are intended, in an alternate embodiment, the cells may be derived from another individual, or from cells lines, or stored blood including stored cord blood, or cells grown from the individual.

Of course, as mentioned above, enhanced heat shock protein uptake for non-immunotherapeutic purposes is fully embraced herein. This may include delivery of, for example, antisense oligonucleotides, vectors, naked DNA, and other macromolecules including pharmaceutical agents to achieve an acute or chronic change in the genotype and optionally the phenotype of the cell.

A cell with increased expression of CD40 exhibits enhanced uptake of heat shock protein or a CD40-binding fragment thereof, which preferably may be a complex with another, pre-selected molecule, as described above. As will be seen herein, such enhanced uptake, in combination with amounts of antigen and various co-stimulatory molecules or in the absence of certain molecules, may induce an effective cellular and/or humoral immune response thereto, or result in tolerizing the immune system to the antigen.

The antigen delivered by a heat shock protein or fragment thereof, such as by natural affinity to the heat shock protein, conjugation to a heat shock protein binding peptide as described above, or covalently bound to the heat shock protein or fragment thereof, of may be an infectious disease antigen or a tumor antigen, by way of non-limiting example. Infectious disease antigens include viral, bacterial, protistan, and parasite antigens, by way of non-limiting example, such as parasitic, fungal, yeast, bacterial, mycoplasmal and viral diseases, where a particular class of cells can be identified as harboring the infective entity. For example, but not by way of limitation, the cells treated may be infected with a human papilloma virus, a herpes virus such as herpes simplex or herpes zoster, a retrovirus such as human immunodeficiency virus 1 or 2, a hepatitis virus, an influenza virus, a rhinovirus, respiratory syncytial virus, cytomegalovirus, adenovirus, Mycoplasma pneumoniae, a bacterium of the genus Salmonella, Staphylococcus, Streptococcus, Enterococcus, Clostridium, Escherichia, Klebsiella, Vibrio, Mycobacterium, to name a few. Protistan antigens include amoeba, malaria and trypanosomal antigens such as those derived from Trypanosoma cruzi. Parasite antigens include schistosomal antigens. Cancer antigens include, for example, those derived from sarcoma, lymphoma, carcinoma, leukemia and melanoma, and include breast carcinoma, ovarian carcinoma, prostate carcinoma, cervical carcinoma, colon carcinoma, lung carcinoma, glioblastoma, and astrocytoma, by way of non-limiting example. Disease-specific epitopes may be found in MHC Ligands and Peptide Motifs, by H. G. Rammensee, J. Bachmann and S. Stevanovic, Landes Bioscience, 1997; and in Rammensee H, Bachmann J, Emmerich N P, Bachor O A, Stevanovic S. SYFPEITHI: database for MHC ligands and peptide motifs. Immunogenetics. 1999 Nov;50(3-4):213-9. These are merely examples of disease-related antigens and epitopes and are not intended to be limiting in any way.

As mentioned above, effective prophylactic or therapeutic immunization for a particular disease may require multiple epitopes or antigens, and the present invention embraces any protocols or compositions which comprise a plurality of epitopes or antigens, such as heat shock proteins isolated from diseased cells for administration in combination with a CD40-modulating protocol as described above, in-vitro-prepared non-covalent complexes of multiple antigens such as peptides, including hybrid peptides as described above, and one or more species of heat shock proteins, and covalent conjugates of a CD40-binding peptide fragment of hsp70 and a plurality of antigens such as peptides, to name a few non-limiting examples.

The foregoing methods are also applicable to in-vivo treatment to upregulate CD40 expression for the purpose of increasing intracellular delivery of heat shock protein.

The foregoing ex-vivo and in-vivo methods may be carried out using a composition or pharmaceutical composition for increasing CD40 expression together with a heat shock protein or complex comprising a heat shock protein, preferably a mammalian and more preferably a human heat shock protein. The invention embraces such compositions comprising a CD40 expression-modulating component and a heat shock protein component, which may be administered to isolated cells from an individual or to the body, included in a pharmaceutically-acceptable formulation.

The mammal used in any of the aforementioned procedures may be any mammal, preferably a human mammal but also including without limitation a domesticated, companion or livestock mammals.

The effective immune response-modulating amount of a complex containing at least a heat shock protein and a pre-selected antigen may be a covalent or noncovalent complex comprising the pre-selected antigen or an immunogenic fragment thereof and the heat shock protein. The heat shock protein is preferably mammalian, more preferably human, most preferably a human hsp70, but it is not so limiting. The complex may be a non-covalent complex of a heat shock protein and a hybrid antigen, the hybrid antigen comprising a covalent conjugate between the pre-selected antigen or an immunogenic fragment thereof and a heat shock protein binding moiety. The heat shock protein binding moiety may be a peptide or an organic molecule, as described in detail above.

Generally, the amount of complex provided to induce tolerization to the antigen is greater than that amount provided to induce an effective immune response thereto. The corresponding doses to achieve these goals may be readily determined. The pre-selected antigen preferably may be an antigen for which tolerizing the immune system is desired, such as an autoimmune antigen, a transplant antigen, an allergen, etc., but it is not so limiting.

In yet a further aspect, a method is provided for enhancing the uptake of heat shock proteins by cells in vivo in an animal by at least administering to the animal an agent capable of increasing CD40 expression by the cells in the animal. In one non-limiting embodiment, the agent may be a vector comprising a polynucleotide encoding CD40. The DNA may be administered using naked DNA or a viral vector, as examples of a wide variety of well-established means to introduce into mammalian cells exogenously-supplied DNA. Construction of such vectors for inducible or constitutive expression are also well-known in the art. In another non-limiting embodiment, the agent may be a calcium ionophore, cytokine or LPS. Means of upregulating CD40 expression by other methods including modulation of transcription factor levels or activities, and/or use of small-molecule agents designed to specifically increase (or decrease; see below) expression of CD40, are fully embraced herein. The in vivo methods of all aspects and embodiments of the invention herein may be carried out locally or systemically, including administration in the vicinity of a diseased tissue or organ, or a solid tumor.

### Decreasing Heat Shock Protein/antigen Complex Uptake by Decreasing CD40 Expression or Binding of Heat Shock Protein to CD40

In still yet a further aspect of the invention, a method is provided for decreasing the uptake of a heat shock protein by a cell expressing CD40 or a cell induced to express CD40 by at least exposing said cell ex vivo to an agent capable of interfering with the binding of said heat shock protein to CD40 expressed by the cell. The agent may be, for example, a CD40 binding partner, such as an antibody or a ligand to CD40. Preferably, the ligand comprises CD40L, such as a soluble form of CD40L. In another embodiment, the ligand may be a CD40-binding portion of a heat shock protein. Preferably, such CD40 ligands are not agonists, i.e., they do not induce signaling by CD40. In a non-limiting example, the CD40-CD40L-antagonistic monoclonal antibody 5D12 described in Boon et al., 2002, Toxicology 174:53-65, and in U.S. Pat. Nos. 5,397,703; 5,677,165 and 6,315,998 maybe used. In another embodiment, the agent may be a CD40 antisense oligonucleotide. Preferably, for this aspect of the invention, the CD40 binding portion of a heat shock protein does not signal. The use of modified forms of the aforementioned agents that do not signal, such as a modified CD40L, may be employed.

### Targeting Molecules to CD40-expressing Cells Using Mammalian Heat Shock Proteins or Fragments, Mimetics, and Enhanced-binding Heat Shock Proteins and Fragments

In yet still a further aspect of the invention, methods are provided for targeting biomolecules such as antigens or drugs to a cell which expresses CD40 or can be modified to express CD40. In one embodiment, such targeting of antigens bypasses the need for a heat shock protein to deliver the antigen, by utilizing a CD40-binding portion of a heat shock protein, or a molecule identified by its ability to modulate the interaction between CD40 and a heat shock protein, this achieving the natural association of the antigen with CD40 as the first step in antigen presentation, without heat shock protein per se but effectively its natural role in the process. Targeting is achieved by utilizing a conjugate or complex between the biomolecule and a heat shock protein fragment which specifically binds to CD40. Such conjugates may be covalent or non-covalent conjugates, and may comprise other components in order to effectively deliver a biomolecule to a CD40-expressing cell. For fragments of native mammalian heat shock proteins, preferably a non-covalent complex between a native heat shock protein fragment and a molecule to be targeted to CD40 is provided should the fragment retain binding activity. In another embodiment, a covalent complex between the CD40-binding heat shock protein fragment and the molecule desirably delivered is preferred. Preferably, the molecule is an antigen or immunogen desirably delivered for the purpose of antigen presentation and eliciting an attendant immune response thereto, whether an elicited humoral and/or cellular immune response, or a decrease of an immune response to the antigen, or tolerizing of the immune system thereto. In a preferred embodiment, the aforementioned method utilizes a covalent conjugate between an antigen or immunogen and a CD40-binding fragment of a heat shock protein, more preferably a fragment of a member of the hsp70 family, and most preferably, human hsp70. The fragment is preferably derived from the N-terminal domain (ATPase domain) of an hsp70 family member. The cell may be induced or otherwise modified as described hereinabove to increase expression of CD40, and such means to increase in expression in combination together with exposing the cells to an antigen complexed with a CD40-binding fragment of a heat shock protein is also embraced herein.

As noted above, a suitable CD40-binding fragment of a mammalian heat shock protein and particularly a hsp70 family member is the N-terminal or ATP-binding (ATPase) domain, extending from about amino acid 1-5 to about amino acid 381 of human hsp70. A suitable but merely exemplary molecule is depicted in SEQ ID NO: 1, which is a 44 kDa fragment of human hsp70, extending from amino acid 5 to amino acid 381, and in SEQ ID NO: 319, which extends from amino acid 1 to amino acid 381 of human hsp70. The invention also embraces fragments of the N-domain that bind to CD40, including fragments as short as about 6 amino acids, by way of non-limiting example. The invention is also directed to compositions and in particular pharmaceutical compositions comprising a covalent or non-covalent conjugate or complex between an antigen or immunogen and a CD40-binding fragment of a mammalian heat shock protein, preferably hsp70, more preferably a mammalian hsp70 and most preferably human hsp70, for the aforementioned uses. The conjugate may be prepared by covalent crosslinking of the two species, using chemical cross-linking agents such as carbodiimides, homobifunctional or heterobifunctional agents (such as are sold by Pierce Chemical Co., Rockford, Ill.). If the antigen or immunogen is a peptide or protein and can be linked to the CD40-binding fragment in a peptide bond, optionally with an intervening linker or spacer sequence, it preferably may be prepared by co-linear synthesis (solid peptide synthesis) or if feasible based on the length of the polypeptide, by expressing the conjugate as a single-chain (or fusion) polypeptide using an expression vector or similar construct. Alternatively, they may be chemically peptide bonded (e.g., by using a carbodiimide). The antigen may be placed at the N-terminal or the C-terminal position relative to the CD40-binding portion, the activity of such constructs readily determinable by preparing the constructs and evaluating them by routine methods. Polynucleotides encoding the aforementioned single-chain polypeptides, including degenerate variants thereof, are likewise embraced herein. Such polynucleotides may be utilized therapeutically to genetically alter cells to express the conjugate, for the purpose of production of molecules for mammalian administration or for in-situ gene therapy purposes.

By way of example, a fusion polypeptide comprises SEQ ID NO: 1 or SEQ ID NO: 319 at the N- or C-terminus, joined through a GSG peptide linker to a melanoma antigen derived from gp 100: 209-217 (210M), (IMDQVPFSVGSGNLLRLTGW; SEQ ID NO: 324) or from tyrosinase: 368-376 (370 D) (YMDGTMSQVGSGNLLRLTGW; SEQ ID NO: 325) at the other end. SEQ ID NOs: 326, 327, 328 and 329 depict hsp70 (1-381)-GSG-gp 100: 209-217 (210M); gp 100: 209-217 (210M)-GSG-hsp70 (1-381); hsp70 (1-381)-GSG-tyrosinase: 368-376 (370 D); and tyrosinase: 368-376 (370 D)-GSG-hsp70 (1-381), respectively. SEQ ID NO: 330 depicts hsp70 (1-381)-GSG-gp 100: 209-217 (210M)-GSG-tyrosinase: 368-376 (370 D), a composition with a single CD40-binding domain and two melanoma epitopes. These are merely exemplary and non-limiting examples of compositions of the invention useful for the purposes mentioned herein.

Furthermore, the aforementioned mammalian heat shock proteins and fragments, in particular the N-domain and fragments thereof, may be altered at the amino acid and/or post-translational level to optimize or enhance binding to CD40. Various mutations in the amino acid sequence, such as but not limited to amino acid substitutions, insertions, deletions, as well as post-translational modifications, such as acetylation, phosphorylation, carbamylation, glycosylation, merely by way of examples, may be performed to provide an enhanced CD40-binding molecule. The present invention embraces all such modifications of heat shock proteins and fragments thereof with enhanced CD40 binding activity, as well as nucleic acids including degenerate sequences that encode such CD40 binding-enhanced heat shock proteins and fragments thereof. Such modified heat shock proteins that include a peptide binding domain may also have increased affinity for peptides in the binding pocket.

Compositions comprising both a CD40 expression upregulator and a conjugate as described above are also embraced here, to enhance uptake. Such upregulators such as calcium ionophores, cytokines and LPS are described above.

### Induction of Signaling in Cells Expressing CD40

In yet another aspect of the invention, means are provided for inducing CD40 signaling in dendritic cells or other CD40-expressing cells. In the practice of this aspect of the invention, a mammalian heat shock protein or a CD40-binding fragment (or enhanced CD40-binding fragments as described above) thereof is used as a ligand for CD40 and thus as an initiator of CD40-mediated signaling in such cells. Such signaling may be used to modulate the maturation of dendritic cells, enhance the development of an immune response in a similar manner as that induced by agonistic anti-CD40 antibodies, among other uses. It may be used in combination with other vaccines or immunogens to enhance the immune response, or under certain conditions, induce tolerance. In addition, the ability for a heat shock protein or fragment thereof to interact with CD40 and induce cytokine production by the cell expressing CD40 is another useful aspect of the invention. SEQ ID NOs: 1 and 319 are non-limiting examples of such compositions.

Molecules embraced herein for the aforementioned purposes include intact heat shock proteins, such as a member of the mammalian hsp70 family, preferably mammalian hsp70, more preferably a human hsp70 family member and most preferably human hsp70. Alternatively, a CD40-signalling fragment of a heat shock protein, such as a fragment of a member of the mammalian hsp70 family, more preferably a fragment of a human hsp70 family member and most preferably a fragment of human hsp70 may be used. The ability of a fragment to induce CD40 signaling is easily determined in vitro. Preferably, the fragment is derived from the N-terminus of ATP-binding (ATPase) domain, and more preferably, derived from about amino acid 5 to about amino acid 381 of hsp70. Most preferably, the fragment is a portion of the aforementioned fragment of the N-terminal domain of hsp70. Examples include SEQ ID NOs: 1 and 319.

### Identification of Heat Shock Protein Mimetics

In another aspect of the invention, the interaction between the N-domain of a mammalian heat shock protein and the exoplasmic domain of CD40 may be advantageously exploited to identify new mimetics of heat shock proteins which have numerous uses, including but not limited to having adjuvant properties as well as being new CD40-targeting agents useful for directing antigens and other biomolecules to cells expressing CD40 or that may be modified to express CD40. Screening methods utilizing the heretofore unexpected interaction between a mammalian heat shock protein 70, in particular its N-domain, and the exoplasmic domain of CD40, may be used in any form to identify compounds or agents that promote or inhibit the interaction, for use in identifying compounds, preferably small-molecule compounds but not being so limited, that would be useful as, for example, targeting agents that when conjugated to an antigen or immunogen, promotes binding to CD40 and uptake, or induces CD40-mediated signaling, the foregoing to promote the induction of an immune response, or, in contrast, inhibits uptake of heat shock protein and any antigen bound thereto by cells expressing CD40, or abrogates CD40 signaling, for the purpose of down-regulating an immune response. The invention embraces the aforementioned methods as well as compounds described above.

The screen may employ native CD40 (SEQ ID NO: 323) and hsp70 (SEQ ID NO: 1), or fragments of either than comprise the interacting regions. Fragments of CD40 include the exoplasmic domain of CD40, that portion of the polypeptide extending from about amino acid 20 to about amino acid 212, and preferably, that portion of the polypeptide extending from amino acid 20 to amino acid 212 (SEQ ID NO: 318). The screen may be carried out with a native mammalian hsp70 family member, preferably a human hsp70 family member, more preferably a human hsp70 family member and most preferably human hsp70, or a CD40-binding fragment of any of the foregoing, preferably the N-terminal or ATP-binding (ATPase) domain, more preferably a polypeptide from about amino acid 5 to about amino acid 381 of human hsp70, and most preferably amino acid 5 to amino acid 381 of human hsp70 (SEQ ID NO: 1). A smaller fragment of either that retain the affinity may be used. Either or both members may be expressed as fusion polypeptides or constructs with a tag for ease in isolation or identification of binding, or as part of any other format for detecting compounds that enhance or inhibit the interaction between a hsp70 and CD40.

As mentioned above, polynucleotides encoding polypeptides or proteins comprising a CD40-binding fragment of a heat shock protein and at least one immunogenic peptide or antigen, and vectors comprising the sequence for expression in bacterial or eukaryotic cells, are useful both for the in-vitro preparation of the aforementioned compositions for mammalian administration, or in another embodiment of the present invention, for administration for therapeutic purposes. Thus, naked DNA or vectors encoding the compositions mentioned above, such as DNA encoding any of SEQ ID NOs: 326-330, may be administered to mammalian patients in need of treatment of melanoma for the purpose of eliciting an immune response. Thus, the invention embraces such polynucleotide sequence encoding a conjugate between a CD40-binding fragment of a heat shock protein and an immunogen, as well as to methods for inducing an immune response for inducing an immune response or treatment of disease.

The amino acid sequences of amino acids 1-381 of human hsp70 (SEQ ID NO: 319) and of amino acids 5-381 of human hsp70 (SEQ ID NO: 1) are merely exemplary of various polymorphisms and variant sequences of the full-length human heat shock protein 70, and the description herein is inclusive of such variations in the human population. Furthermore, the fragment of CD40 extending from amino acid 20 to 212 (SEQ ID NO: 318) is also inclusive of polymorphisms and other variations in the sequence among the population, and this aspect of the invention fully embraces such variant molecules.

The invention may be appreciated by the following examples, which are merely illustrative of certain aspects of the invention and are non-limiting.

### EXAMPLE 1

### Materials and Methods

Plasmids and protein preparation. Human Hsp70, its N-terminal (N70) and C-terminal (C70) domain, Hip, and Bag-1 were expressed and purified as His-tagged proteins (Scheufler et al., 2000, Cell 101, 199-210) (Sondermann et al., 2000, Biol Chem 381, 1165-74; Hohfeld et al., 1995, Cell 83, 589-98). The mycobacterial hsp70 homologue DnaK was expressed as described (Szabo et al., 1994, Proc Natl Acad Sci USA 91, 10345-9). Equine glutathione-S-transferase (GST) was purchased from SIGMA (Germany). Biotinylated recombinant Hsp70 and equine GST were prepared by labeling with the FluoReporter Biotin-XX Protein Labeling Kit (Molecular Probes). Human CD40 cDNA was amplified by PCR from a human primary macrophage cDNA library (Invitrogen), and inserted into pcDNA3.1/Zeo and pIRES2-EGFP vector (Invitrogen). The cDNA for the exoplasmic domain of CD40 (amino acids 20-212; SEQ ID NO: 318) was amplified by PCR from the human CD40 cDNA described above, and inserted into pGEX-2T vector (Amersham Pharmacia). GST and GST-CD40 fusion protein was purified by glutathione affinity chromatography (Glutathione Sepharose 4 Fast Flow, Amersham Pharmacia). Peptide C (GCEVFGLGWRSYKH; SEQ ID NO: 320), biotinylated peptide C (biotin-GCEVFGLGWRSYKH; SEQ ID NO: 321) and peptide C-FITC (FITC-GCEVFGLGWRSYKH; SEQ ID NO: 322) were custom synthesized by R. Piepkom (Deutsches Krebsforschungs Zentrum, Heidelberg).

For Hsp70/peptide complex formation, Hsp70 was incubated with a 5-fold excess of peptide C, biotinylated peptide C or peptide C-FITC for 30 min at 37° C. in binding buffer (10 mM MOPS/KOH, pH 7.2,150 mM KCl, 2 mM ADP and 3 mM MgCl₂). Excess of unbound peptide C-biotin was removed by gel filtration on Sephadex G-50 (Amersham Pharmacia). Quantification of Hsp70 complex formation by ELISA using a streptavidin-peroxidase conjugate (Molecular Probes) indicated yields of 20-30%.

All proteins were centrifuged at 100,000 g at 4° C. for 1 hour prior to the experiments in order to remove aggregates.

Cell culture and fluorescence microscopy. The murine macrophage cell line ANA1 was kindly provided by H. Wagner (Munich, Germany). ANA-1 cells were cultured in VLE-RPMI 1640 (Biochrom), supplemented with 10% FCS, 2 mM L-glutamine and antibiotics. For fluorescence microscopy, ANA1 cells were either stimulated with LPS (20 μg/ml, Sigma, Germany), or kept in an LPS-free medium (mock treatment) for 7 h. After stimulation, cells were harvested and incubated with 100 nM biotinylated Hsp70, biotinylated GST, or Hsp70/peptide C-biotin complex for 30 min at 0° C. in VLE-RPMI 1640. After incubation cells were processed for staining with streptavidin-TRITC (Sigma, Germany), fixed with paraformaldehyde and analyzed by fluorescence microscopy (Axiovert 35, Zeiss) as described (Sondermann et al., 2000). Cos-7 cells were cultured in DMEM, supplemented with 10% FCS, 2 mM L-glutamine and antibiotics (Biochrom), grown on cover slips in 24 well-plates and transiently transfected with human CD40 cDNA (inserted into the pIRES2-EGFP vector (Invitrogen; see Example 3)) by the calcium phosphate method (Chen and Okayama, 1987). Twenty-four h post-transfection cells were incubated with 250 nM biotinylated Hsp70, biotinylated GST or Hsp70/peptide C-biotin complex for 30 min at 0° C. in DMEM, fixed, stained with streptavidin-TRITC (Sigma, Germany), and analyzed by fluorescence microscopy as described (Sondermann et al., 2000, idem). HEK293T cells were grown on collagen coated dishes or cover slips, and cultured in DMEM, supplemented with 10% FCS, 2mM L-glutamine and antibiotics. HEK293T cells were stably transfected (Chen and Okayama, 1987, Mol Cell Biol 7, 2745-52) with CD40 cDNA cloned into pcDNA3.1/ZEO (Invitrogen), and selected with Zeocin (Invitrogen). Stably transfected cell lines were analyzed by immunoblotting for CD40 expression using an anti-CD40 antibody (CSA180, Stressgen). HEK293T-MCAT cells expressing the murine cationic amino acid transporter (MCAT) cloned into pcDNA3.1/Zeo vector were kindly provided by W. Nickel (Heidelberg, Germany). S-Hela cells were cultured in alpha-MEM supplemented with 8% FCS, L-glutamine and antibiotics, and grown in spinner-flasks.

For experiments to study uptake of Hsp70/peptide C-FITC complex, HEK293T-CD40 and HEK293T-MCAT cells were seeded on collagen-coated cover slips 24 h prior to the experiment. For complex formation Hsp70, its N-terminal domain (N70) or its C-terminal domain (C70) were preincubated with a 10-fold molar excess of peptide C-FITC for 30 min at 37° C., as described above for the biotinylated proteins. HEK293T-CD40 and HEK293T-MCAT were incubated with 0.5 μM of Hsp70, N70 or C70, preincubated with 2 mM ADP and 5 μM peptide C-FITC, or 5 μM peptide C-FITC alone for 30 min on ice. Thereafter the cells were washed three times with medium, incubated for 15 min at 37° C., and fixed, embedded in Fluoromount-G and analyzed by confocal microscopy (LSM 510, Zeiss).

Analysis of CD40 expression. For immunoblot analysis ANA1 cells were stimulated with LPS or mock treated as described above, harvested, lysed by repeated passage through a needle (0.4-0.8 mm, Braun) in the presence of protease inhibitors (Complete, EDTA free, Roche), and fractionated by centrifugation at 100,000 g at 4° C. for 1 hour. The pellet was resuspended in 0.1% Triton X-100/PBS, and protein concentrations determined in total lysate, supernatant and pellet fractions by the Bradford assay (Biorad). Equal amounts of protein were subjected to SDS-PAGE, and analyzed by immunoblotting with an anti-CD40 antibody (CSA180, Stressgen).

Binding assays with GST-CD40. For binding of endogenous Hsp70 and Hsc70 from Hela cell lysate, Hela cells were lysed as described above and centrifuged at 100,000 g at 4° C. for 1 h. 20 μl bed volume of Glutathione Sepharose 4 Fast Flow (Amersham Pharmacia) per sample were preincubated with 80 pmol or 400 pmol of GST or GST-CD40 fusion protein in 50 μl binding buffer. Unbound protein was removed by washing three times with PBS. The beads were treated with 1% BSA/PBS to avoid non-specific binding, and than incubated with 150 μl of Hela cell lysate in the presence of 2 mM DTT for 20 min at 16° C. The immobilized proteins were washed three times with 1 ml PBS and eluted with 20 μl elution buffer (50 mM Tris/HCl, pH 8.0, 10 mM reduced glutathione) for 10 min at 37° C. 10 μl of the eluates was subjected to SDS-PAGE and analyzed by immunoblotting using an anti-Hsc/Hsp70 antibody and an anti-Hsp90: antibody (SPA822 and SPA835, Stressgen).

For binding assays with recombinant Hsp70 and its domains, 3 μM His6-Hsp70 or its His6-tagged domains were incubated with 2 mM ATP, 2 mM ADP or 2 mM ADP and 28 μM peptide C in 50 μl binding buffer (10 mM MOPS/KOH, pH 7.2, 150 mM KCl and 3 mM MgCl2) for 30 min at 37° C. In competition experiments binding was probed with either a 10-fold excess of Hsp70, the two domains, or DnaK, or with a 5-fold excess of Hip or Bag 1 for 10 min at 0° C. To this end, Hsp70, N70, C70 and DnaK were preincubated with ADP, ATP and peptide C as described above. For comparison of CD40 binding of Hsp70 and its N-terminal domain, Hsp70 was preincubated in the presence or absence (mock treatment) of a 30-fold molar excess of peptide C. 3 μM of GST or GST-CD40 was added to the samples and incubated for 20 min at 16° C. For titration of peptide C, 5- to 50-fold excess of peptide C (15-150 μM) was added to the preincubation of Hsp70. Thereafter the samples were combined with 20 μl of 1% BSA-treated Glutathione Sepharose 4 Fast Flow and incubated in the presence of 2 mM DTT for another 20 min at 16° C. Unbound proteins were removed by washing the beads three times with 1 ml PBS, and immobilized proteins eluted with 30 μl elution buffer for 20 min at 22° C. 10 μl of the eluates were subjected to SDS-PAGE and analyzed by immunoblotting using a Penta-His-antibody (Quiagen). Blot signals were quantified by Quantity One (Biorad).

Binding assay with His6-tagged Hsp70 and Ni-NTA-agarose. ANA1-cells were stimulated with LPS as described above and lysed in lysis buffer (150 mM Tris/HCl, ph 7,5, 1% CHAPS) for 45 min at 4° C. Cell lysate was centrifuged at 100,000 g for 15 min at 4° C. 500 l of the supernatant was incubated with 10 μg of His6-tagged Hsp70, preincubated with ADP and a 30-fold excess of peptide C, or mock treated, as described above for biotinylated proteins, for 30 min at 4° C. Thereafter the samples were added to 20 μl of 1% BSA-treated Ni-NTA-agarose (Quiagen) and incubated for 30 min at 4° C. Unbound protein was removed by washing the beads three times with 1 ml lysis buffer, and immobilized proteins eluted with 10 μl SDS-PAGE-sample buffer by incubation for 5 min at 95° C. Eluates were analyzed by immunoblotting using an anti-CD40 antibody (CSA-180, Stressgen).

P38 Kinase Assay. Twenty-four h prior to P38 kinase assays HEK293T-CD40 and HEK293T-MCAT cells were seeded into a 24-well plate coated with collagen and incubated with 100 nM CD40L (Alexis), HSP70, N70-domain, C70-domain, or DnaK for 20 min at 37° C. DnaK, Hsp70, N70 and C70 were preincubated with either 2 mM ADP and 30 μM peptide C or 40 μM AMPPNP (Sigma). Upon stimulation cells were washed twice with ice-cold PBS, and lysed in SDS-sample buffer. Equal amounts of protein were analyzed by immunoblotting with antibodies directed against phosphorylated P38 (Promega), and with a monoclonal anti-tubulin antibody (J. Wehland, Braunschweig, Germany).

### EXAMPLE 2

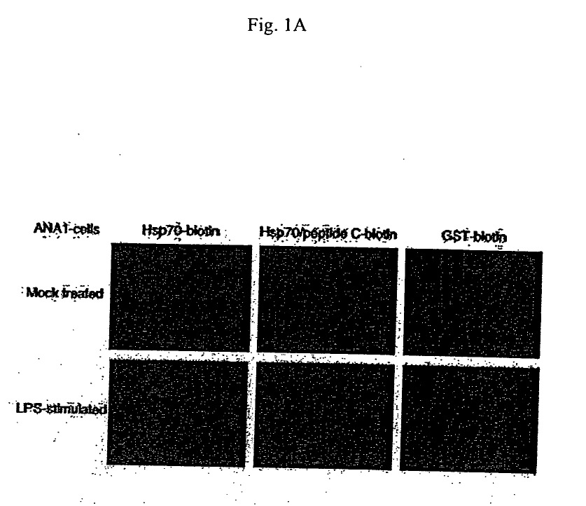

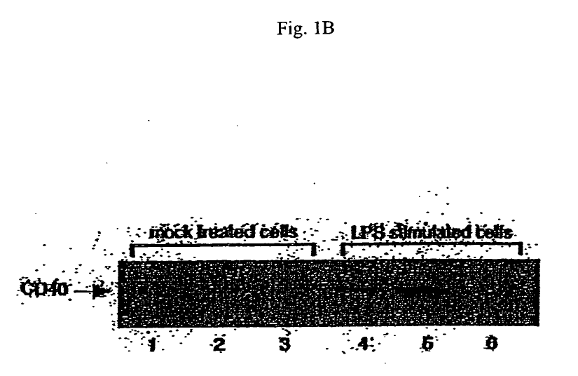

In order to determine the molecular basis for the binding of human Hsp70 to ANA-1 cells (Sondermann et al., 2001, Science 291, 1553-7), cells were incubated either with biotinylated Hsp70 or with Hsp70 that had been loaded with biotinylated peptide C (GCEVFGLGWRSYKH; SEQ ID NO: 320; Flynn et al., 1989, Science 245, 385-90) in the presence of ADP. Bound protein was detected by fluorescence microscopy after reaction with fluorescent streptavidin (see Example I). Unstimulated cells showed weak but clearly detectable binding with both Hsp70 and Hsp70-peptide complex, whereas no binding was observed with biotin-labeled glutathione S transferase (GST) as a control protein (FIG. 1A, upper panels). In contrast, LPS-stimulation resulted in a significant increase in binding of Hsp70 and Hsp70-peptide, but not of GST (FIG. 1A, lower panels).

Details for FIG. 1. Lipopolysaccharide (LPS) treatment of ANA1-cells stimulates binding of Hsp70 and induces expression of CD40. Cells were incubated with LPS or mock treated as outlined in Example I. A. Cells were incubated either with biotinylated Hsp70, Hsp70 loaded with biotinylated peptide C, or with biotinylated GST as a control. After 30 min at 4° C. cells were washed, incubated with TRITC-labeled streptavidin, washed again, and processed for fluorescence microscopy. Upper panels: mock treated ANA1-cells; lower panels: ANA1-cells after treatment with LPS. Note that that Hsp70 carries on average 5 biotins, whereas peptide C contains a single biotin. B. Cells were harvested, lysed and centrifuged to obtain a total membrane pellet. Identical protein amounts of the samples were analyzed by immunoblotting with an antibody directed against CD40. Lanes 1 and 4: total cell lysates; lanes 2 and 5: membrane fractions; lanes 3 and 6: supernatants.

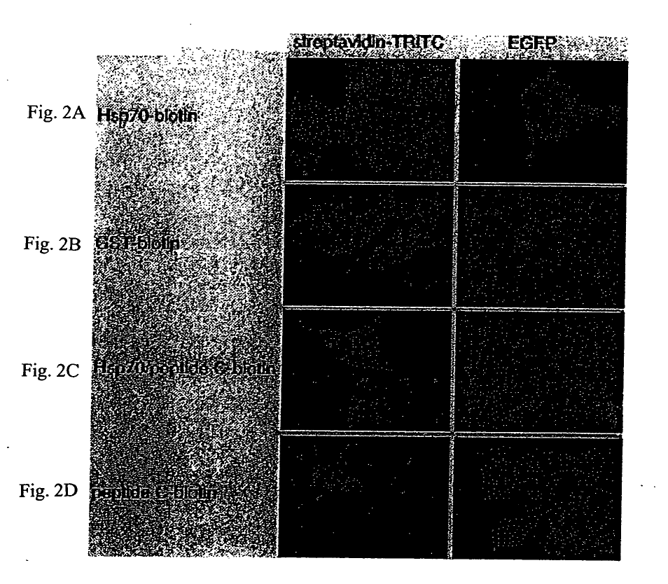

Mock-treated and LPS-stimulated ANA-1 cells were analyzed for their expression of CD40. As shown in FIG. 1B, LPS-stimulated cells, but not control cells, express high levels of membrane-bound CD40, as detected by immunoblotting (Tone et al., 2001, Proc Natl Acad Sci USA 98, 1751-6). In order to assess the possibility that CD40 itself may serve as an Hsp70 receptor, Cos-7 cells, which do not express CD40, were transfected with a cDNA construct containing the cDNA for human CD40, followed by an internal ribosomal entry site and the cDNA for EGFP. Transfected cells were identified by virtue of their GFP fluorescence, and were analyzed for their ability to bind biotin-labeled Hsp70 or Hsp70-biotin-peptide complex (FIG. 2).

Indeed, Hsp70 and its peptide complex bound to the surface of transfected cells but not to untransfected cells, whereas no binding above background was detected with biotinylated GST or biotinylated peptide alone (FIG. 2).

Details for FIG. 2. Transfection with human CD40-cDNA renders Cos-7 cells active in binding human Hsp70. Cos-7 cells were transiently transfected with a fusion construct that contains human CD40-cDNA, followed by an internal ribosomal entry site and the cDNA for EGFP, giving rise to green fluorescence of transfected cells. Cells were then incubated for 30 min at 0° C. with biotinylated Hsp70 (A), biotinylated GST as a control (B), Hsp70-peptide complex containing biotinylated peptide C (C), or with biotinylated peptide C alone (D). Thereafter cells were washed and incubated with TRITC-streptavidin and processed for fluorescence microscopy as described in FIG. 1. Left hand panels show streptavidin-fluorescence and right hand panels EGFP-fluorescence.

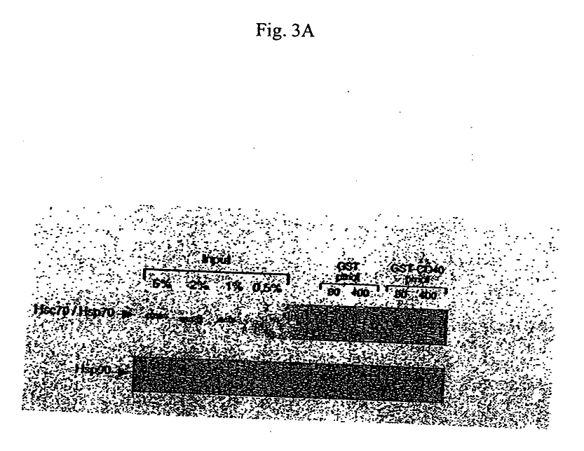

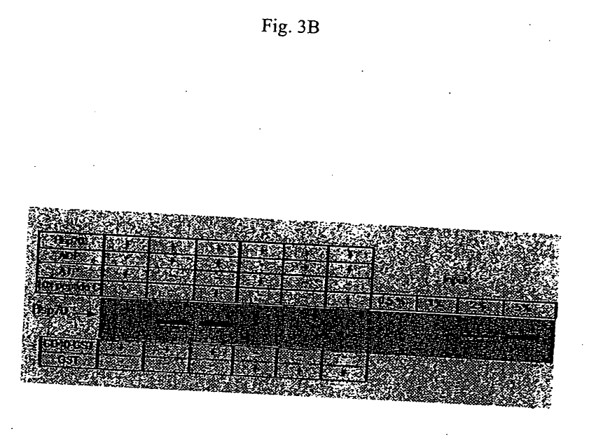

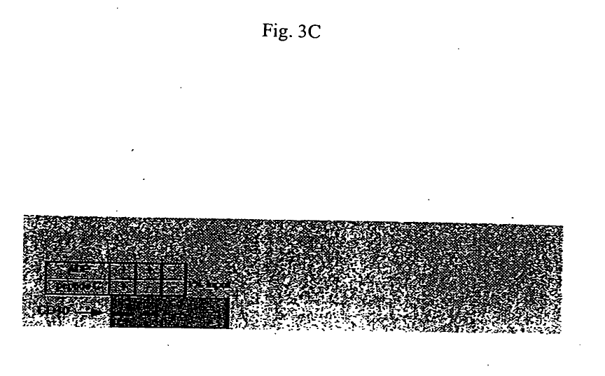

Binding experiments were performed in vitro to determine whether CD40 and Hsp70 interact directly. To this end, the exoplasmic domain of human CD40 (amino acid residues 20-212) was expressed in E. coli as a soluble GST-fusion protein and used to study its interaction with heat-shock proteins in Hela-cell lysates. Increasing amounts of GST-CD40 were incubated with the lysates and bound proteins were adsorbed to glutathione-Sepharose, followed by immunoblotting with antibodies directed against Hsc70, Hsp70 and Hsp90. As shown in FIG. 3A, Hsc70 and Hsp70 bound to GST-CD40 in a concentration dependent manner, whereas Hsp90 did not interact. As will be shown later, the interaction of Hsp70 with CD40 is enhanced in the presence of ADP. GST alone, used as a negative control, did not bind Hsc70/Hsp70. These experiments demonstrate a direct and specific interaction of CD40 with mammalian Hsp70 family members.

Human Hsp70 binds to CD40 via its ATPase domain. Next, we investigated the biochemical requirements for CD40-Hsp70 complex formation. Specifically, we addressed the question whether Hsp70 binds to CD40 via its C-terminal substrate-binding domain, or via its N-terminal nucleotide binding domain. In this context it was of interest whether the association depended on a specific nucleotide-bound state of Hsp70. Recombinant Hsp70 was incubated with GST-CD40 in the presence of ADP or ATP, with or without substrate peptide (FIG. 3B). Hsp70 binding to CD40 was barely detectable in the presence of ATP, and was only seen in the presence of ADP. Notably, addition of a molar excess of peptide C over Hsp70 did not compete with the interaction, as might have been expected if the C-terminal Hsp70 domain were to mediate binding of Hsp70 to CD40. Only background signals were observed when GST alone was analyzed as a control. Recombinant, His-tagged Hsp70 also interacted, in an ADP-dependent manner, with endogenous CD40 in extracts of LPS-stimulated ANA1-cells, as detected by adsorptions of the complex to Ni-NTA-agarose and immunoblotting with CD40 antibody (FIG. 3C). Together these results suggested that Hsp70 binds to CD40 via its ATPase domain.

Details for FIG. 3. Binding of Hsp70 to CD40 is direct and depends on ADP. A. Hela-cell lysates were incubated with GST (control), or GST-CD40 in the amounts indicated. Thereafter samples were affinity purified on glutathione-Sepharose as outlined in Experimental Procedures, subjected to SDS-PAGE and analyzed by immunoblotting with antibodies directed against Hsc70 and Hsp70 (upper lanes), and against Hsp90 (lower lanes). B. Human recombinant His6-tagged Hsp70 was incubated with ATP, ADP, or an excess of peptide C, followed by addition of GST-CD40 or GST alone. After affinity purification samples were analyzed by immunoblotting as described in A, using antibodies directed against the His6-tag. C. Recombinant human His6-tagged Hsp70 was incubated with peptide C, either in the presence or absence of ADP. The reactions were then incubated with ANA1-cell lysates as described in Experimental Procedures. Protein bound to Hsp70 was analyzed by affinity purification on Ni-NTA-agarose and immunoblotting with antibodies directed against CD40.

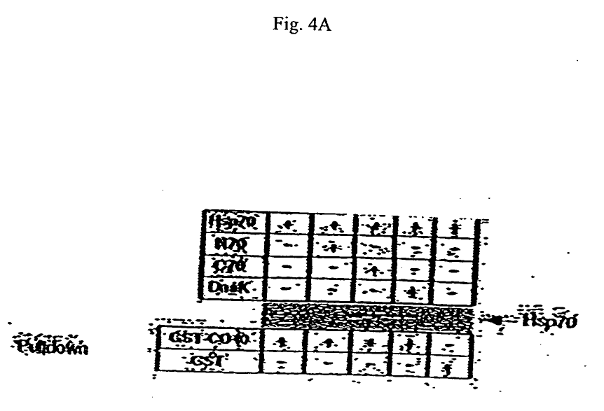

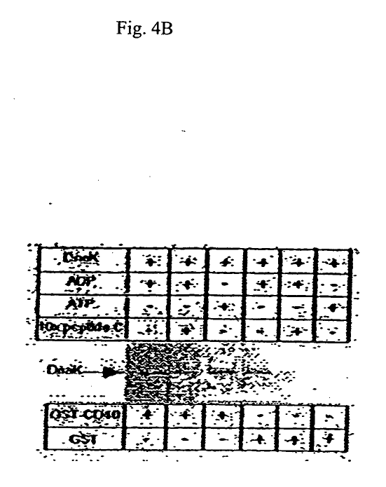

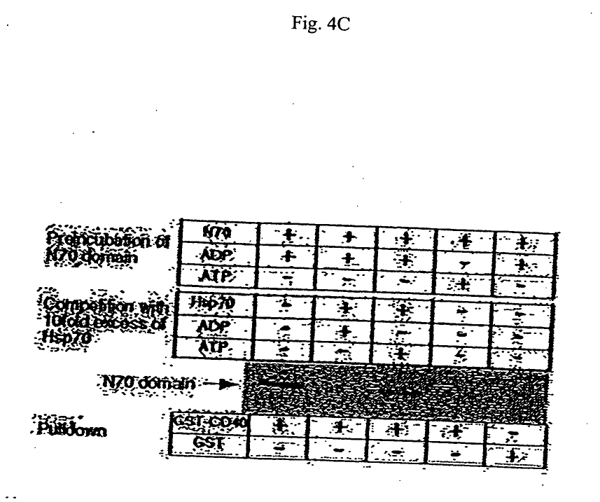

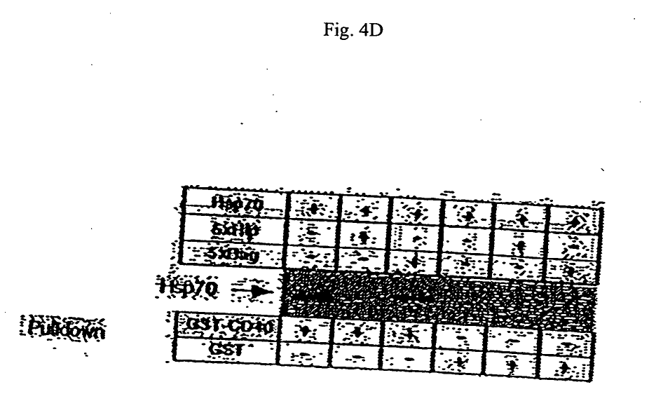

In light of recent findings that the bacterial Hsp70 homologue DnaK associates with CD40 via the C-terminal, peptide-binding domain (Wang et al., 2001, Immunity 15, 971-83), experiments were performed to directly determine the specificity of CD40 for the domains of human Hsp70. Recombinant, full-length Hsp70 was incubated in the presence of ADP and a 5-fold molar excess of either recombinant Hsp70 N-terminal domain (residues 5-381) (Sondermann et al., 2001, idem) or C-terminal domain (residues 382-641) (Scheufler et al., 2000, Cell 101, 199-210), or of recombinant, full-length DnaK. The N-terminal domain of Hsp70 (N70) efficiently competed for the binding of full-length Hsp70 to CD40, as detected with an antibody against the N-terminal 6His-tag on recombinant Hsp70 (FIG. 4A). The ATPase domain itself bound to CD40 in the presence of ADP but not ATP (FIG. 4B). In contrast, neither the C-terminal Hsp70 domain (C70) nor DnaK had a significant effect on binding (see FIG. 4A). In order to analyze whether under this condition DnaK binds directly to CD40, a pull down experiment was performed with DnaK. As shown in FIG. 4B, DnaK indeed binds to CD40, and this interaction is enhanced in the presence of ADP when compared with ATP. This establishes that Hsp70 and DnaK bind to different sites on CD40, and confirms that DnaK binds via its C-terminal domain as reported earlier (Wang et al., ibid.) This binding strongly suggested that it is the substrate-binding site of DnaK that mediates the interaction with CD40. To address this possibility, a competition experiment was performed with an excess of peptide C. As shown in FIG. 4B, a 10-fold molar excess of peptide C almost completely abolishes the binding of DnaK to CD40. Thus, it is the substrate-binding site of DnaK that interacts with CD40, in contrast to the ATP-binding domain (ATPase domain) of mammalian heat shock proteins. In summary, Hsp70 interacts with CD40 dependent on ADP, and via its ATPase domain, as shown with the endogenous proteins in cell extracts and the recombinant proteins in vitro. In contrast, DnaK binds to CD40 via its substrate-binding site proper.

Details for FIG. 4. Hsp70 binding to CD40 is mediated by the N-terminal ATPase domain and is competed by Hip. A. Human His6-tagged Hsp70, its N- or C-terminal domains, or recombinant bacterial DnaK was incubated either with GST-CD40, or with GST. B. Recombinant DnaK was incubated with ATP, ADP, or an excess of peptide C, followed by addition of GST-CD40 or GST alone. C. His6-tagged N-terminal domain of Hsp70 (N70) was incubated in the presence of ATP or ADP, followed by incubation with a 10-fold molar excess of Hsp70 in the presence of ADP or ATP. D: Recombinant human His6-tagged Hsp70 protein was incubated with a 5-fold molar excess of either recombinant Hip protein or Bag-1, and with GST-CD40 or GST as a control. Bound protein was analyzed after affinity purification on glutathione-Sepharose by immunoblotting with an antibody directed against the N-terminal His6-tags or with an antibody directed against DnaK.

Hsp70 binding to CD40 is competed by the Hsp70 cochaperone Hip and is enhanced by peptide substrate. The N-terminal domain of Hsp70 is known to specifically interact with the regulatory cochaperone proteins Hip and Bag-1. While Hip stabilizes the ADP-state of Hsp70 which binds substrate tightly (Hohfeld et al., 1995, ibid), Bag-1 functions as an ADP-ATP exchange factor and causes substrate release from Hsp70 (Hohfeld and Jentsch, 1997, EMBO J 16, 6209-16; Sondermann et al., 2001, idem). In light of the observed ADP-dependence, it seemed possible that CD40 binding to Hsp70 has certain features in common with the interaction between Hip and Hsp70. Binding of Hsp70 to CD40 was analyzed in the presence of a 5-fold molar excess of recombinant Hip or Bag-1 over Hsp70 (FIG. 4D). Hip acted as an effective competitor of the interaction, in contrast to Bag-1, which interacts only relatively weakly with the ADP-state of Hsp70 (Hohfeld, 1998, Biol Chem 379, 269-74; Sondermann et al., 2001, idem). Thus, CD40 and Hip may share similar binding regions on the ATPase domain of Hsp70.

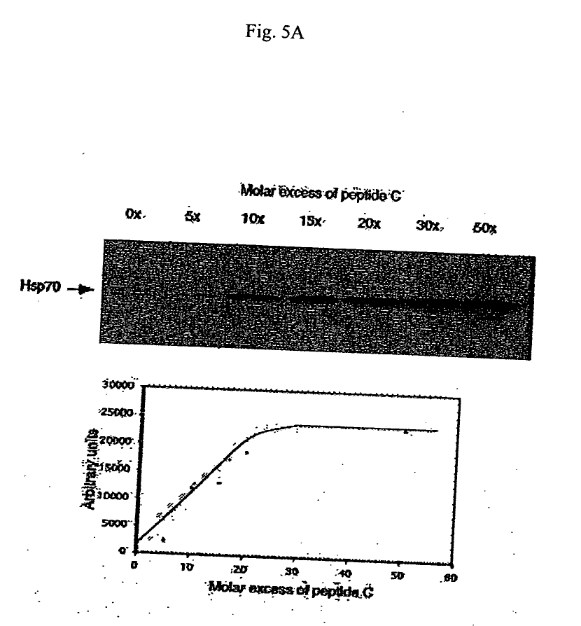

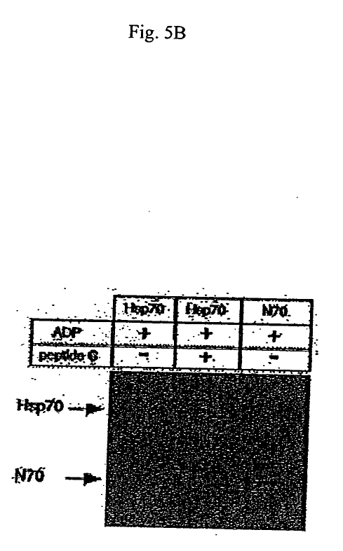

The known cochaperone function of Hip in stabilizing Hsp70 in its substrate-bound ADP-state (Hohfeld et al., 1995, idem) raised the interesting possibility that the interaction between CD40 and Hsp70 may not only be ADP-regulated but may also depend on Hsp70 substrate. Strikingly, binding of Hsp70-ADP to CD40 increased dramatically in the presence of peptide C (FIG. 5A and B). The effect of peptide was saturable (ka˜30 μM), in a range corresponding to the affinity of peptide C for Hsp70 (5-10 micromolar, Greene et al., 1995, J Biol Chem 270, 2967-73). These results demonstrate that it is the peptide- and ADP-bound state of Hsp70 that is recognized preferentially by CD40 and suggest that, similar to Hip, CD40 has a cochaperone-like regulatory function in stabilizing the Hsp70-substrate complex. This effect would ensure that CD40 binds Hsp70 predominantly when it is in complex with peptide.

Details for FIG. 5. Peptide substrate stimulates Hsp70 binding to CD40. A. His6-tagged Hsp70 was incubated with increasing concentrations of peptide C as indicated in the Figure, and binding to GST-CD40 analyzed by immunoblotting with antibodies directed against the His6-tag. The lower panel is a quantitation of the data. B. Equivalent concentrations (3 μM) of His6-tagged Hsp70 (in the presence of a 30-fold molar excess of peptide C) or His6-tagged N70 was incubated with GST-CD40, and bound protein was analyzed as described above.

The striking increase in CD40 binding of ADP-loaded Hsp70 observed in the presence of peptide substrate raised the question whether the free C-terminal domain of Hsp70 has an inhibitory effect on the ATPase domain, reducing the ability of the latter to recognize CD40. This possibility was addressed by comparing the binding efficiency of substrate-saturated Hsp70 to that of the N-terminal domain, both in their ADP states. As show in FIG. 5C, substrate-loaded Hsp70 and the N-terminal domain bound to CD40 with similar efficiencies, indicating that the peptide-free C-terminal domain of Hsp70 masks the N-domain for binding to CD40, either directly or by causing an allosteric conformational change.

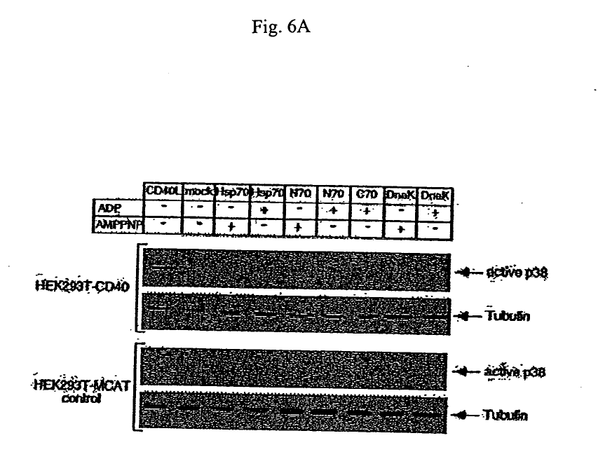

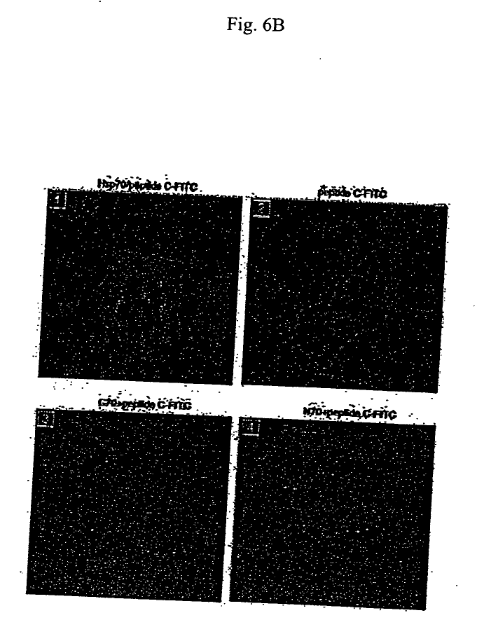

Binding of Hsp70-peptide complex to CD40 results in intracellular signaling and peptide uptake. Binding of CD40 ligand to CD40 induces signal transduction via phosphorylation of p38, a component of the signal cascade between activated CD40 and NF-kappaB, which eventually results in the release of TNF-alpha and subsequent secretion of interferon-gamma (Pullen et al., 1999). Binding of the C-terminal domain of DnaK to CD40 was reported to have a similar effect (Wang et al., 2001, idem). We therefore investigated whether binding of human Hsp70-peptide complex to CD40 also stimulates this signaling pathway. These experiments were carried out in HEK293T cells stably transfected with human CD40 cDNA. After incubation with either Hsp70-peptide complex, recombinant Hsp70 domains, or DnaK, in the presence of ADP or the non-hydrolysable ATP analogue AMPPNP, cells were lysed, and lysates analyzed by immunoblotting with an antibody directed against active (i.e., phosphorylated) p38. Indeed, human Hsp70 and its ATPase domain caused an increase in phosphorylated p38 to an extent comparable to that observed with DnaK (FIG. 6A, upper panel). Although modest when compared to the signal induced by the same molar concentration of CD40 ligand, activation of p38 by Hsp70 was significant, and depended on the presence of ADP. As a control, HEK293T cells stably transfected with the same vector but containing the cDNA for an unrelated membrane protein (murine cationic amino acid transporter, MCAT), did not show a detectable response to the various stimuli (FIG. 6A, lower panel). Thus, Hsp70-peptide complex and the Hsp70 ATPase domain activate signaling via CD40 dependent on the presence of ADP and in a manner comparable to the effect of DnaK, although the latter binds to CD40 via its C-terminal domain (Wang et al. 2001, idem).

Next, the binding of Hsp70-peptide complex to CD40 was studied, which results in the uptake of peptide. To this end, HEK293T-CD40 cells and HE293T-MCAT cells (not expressing CD40) were incubated with a fluorescent (FITC) derivative of peptide C for 30 min at 0° C. in the presence of ADP, with or without Hsp70 or its two domains. After removal of excess material, the cells were incubated at 37° C. for 15 min, fixed and then analyzed by fluorescence microscopy. Representative images are presented in FIG. 6B. After 15 min incubation at 37° C., fluorescent peptide was observed in punctuate intracellular structures only in cells expressing CD40, and only when Hsp70 was present during the incubation for binding at 0° C. (FIG. 6B, panel 1). No staining above background was detected when Hsp70 was omitted (FIG. 6B, panel 2). Likewise, incubation of HEK293T-CD40 cells with either recombinant C70 or N70 and peptide did not result in a peptide signal above background (FIG. 6B, panels 3 and 4). Neither did HEK293T-MCAT control cells give rise to a signal under any of the conditions tested (not shown). We conclude that the specific interaction of CD40 with Hsp70-peptide complex mediates the uptake of peptide.

Details for FIG. 6. Binding of Hsp70 complex to CD40-expressing HEK293T cells induces signaling via p38 and causes peptide uptake. A. HEK293T-cells, stably transfected with cDNA encoding human CD40 or the unrelated membrane protein murine cationic amino acid transporter (MCAT) were incubated for 20 min at 37° C. with CD40L or with Hsp70, C70, N70, bacterial DnaK either in the presence of ADP or AMPPNP, or buffer alone. Thereafter cells were washed, solubilized in SDS-sample buffer, and analyzed by immunoblotting with antibodies directed against phosphorylated (active) p38. Blots were also developed with an antibody against tubulin in order to control for equal loading. B. Cells were incubated at 0° C. for 30 min with recombinant human Hsp70, N70, or C70, all in the presence FITC-labeled peptide, or with the FITC-labeled peptide alone. Thereafter cells were washed, incubated for 15 min at 37° C., and processed for fluorescence microscopy. Upper left panel: Hsp70, upper right panel: FITC-labeled peptide alone, lower left panel: C70, lower right panel: N70. Only cells stably transfected with CD40 are shown. Cell boundaries are emphasized with broken lines. MCAT-expressing control cells did not show fluorescence above background.

### EXAMPLE 3

### Human CD40-IRES-EGFP Construct

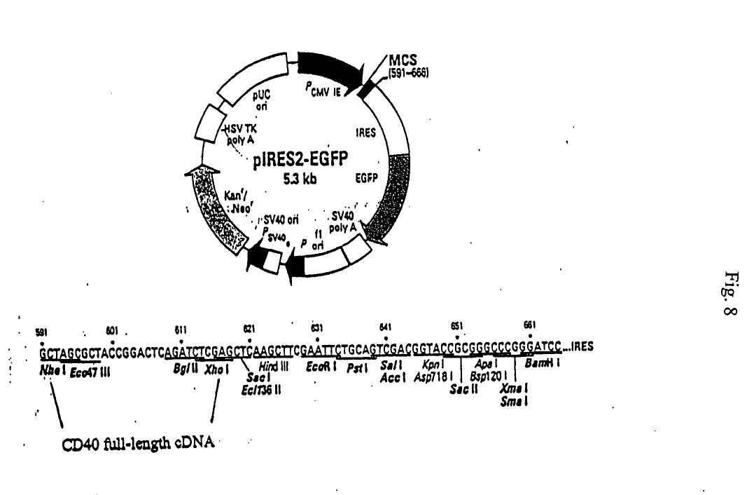

CD40 full-length cDNA was cloned by PCR. A premade cDNA library with human macrophages as the RNA source (Invitrogen) was used as a template for the PCR. The PCR product was made with a 5′-Nhel and a 3′-Xhol restriction site. The PCR product was cloned for sequencing into a pcDNA3.1 vector (Invitrogen) and for mammalian expression studies into a pIRES2-EGFP vector (Clontech). FIG. 8 shows pIRES2-EGFP vector information.

All documents cited throughout are incorporated herein by reference in their entireties.

What is claimed is:

1. A method for enhancing the binding to a cell of a complex comprising a mammalian heat shock protein and a molecule bound thereto, said method comprising increasing CD40 expression by said cell.

2. The method of claim 1 wherein said mammalian heat shock protein is a hsp70 family member.

3. The method of claim 1 wherein said cell is exposed to a CD40-expression-increasing amount of an expression vector comprising a polynucleotide encoding CD40 or a heat shock protein binding fragment of CD40.

4. The method of claim 1 wherein said cell is exposed to a CD40-expression-increasing amount of a calcium ionophore, a cytokine, or LPS.

5. The method of claim 4 wherein said cytokine is IL-1 alpha, TNF-alpha, IFN-gamma, IL-3 or GM-CSF.

6. The method of claim 1 wherein said complex comprising a mammalian heat shock protein and a molecule bound thereto is a covalent or noncovalent complex comprising an antigen and said heat shock protein.

7. The method of claim 6 wherein said noncovalent complex comprises a heat shock protein and a hybrid antigen, said hybrid antigen comprising a covalent conjugate between said antigen and a heat shock protein binding moiety.

8. The method of claim 7 wherein said heat shock protein binding moiety is a peptide or an organic molecule.

9. The method of claim 1 wherein said cell is a professional antigen presenting cell or a non-professional antigen presenting cell.

10. The method of claim 1 wherein said professional antigen presenting cell is a dendritic cell.

11. The method of claim 6 wherein said antigen is an infectious disease antigan or a tumor antigen.

12. The method of claim 1 wherein said increasing is carried out in vitro, ex vivo or in vivo.

13. A method for enhancing the uptake into a cell of a pre-selected molecule comprising exposing said cell to an agent that increases CD40 expression by said cell, and exposing said cell to a complex comprising said pre-selected molecule and a mammalian heat shock protein or a CD40-binding fragment thereof.

14. The method of claim 13 wherein said mammalian heat shock protein is a hsp70 family member.

15. The method of claim 13 wherein said agent is an expression vector comprising a polynucleotide encoding CD40 or a heat shock protein binding fragment thereof.

16. The method of claim 13 wherein said agent is a calcium ionophore, a cytokine, or LPS.

17. The method of claim 16 wherein said cytokine is IL-1 alpha, TNF-alpha, IFN-gamma, IL-3 or GM-CSF.

18. The method of claim 13 wherein said heat shock protein is a covalent or noncovalent complex comprising said pre-selected molecule and said heat shock protein.

19. The method of claim 18 wherein said non-covalent complex comprises a heat shock protein and a hybrid antigen, said hybrid antigen comprising a covalent conjugate between said pre-selected molecule and a heat shock protein binding moiety.

20. The method of claim 19 wherein said heat shock protein binding moiety is a peptide or an organic molecule.

21. The method of claim 13 wherein said cell is a professional antigen presenting cell or a non-professional antigen presenting cell.

22. The method of claim 13 wherein said professional antigen presenting cell is a dendritic cell.

23. The method of claim 18 wherein said pre-selected molecule is an infectious disease antigen or a tumor antigen.

24. The method of claim 13 wherein said exposing to said agent and said exposing to said complex are independently selected from the group consisting of in vitro, ex vivo and in vivo.

25. A method for enhancing the development in a mammal of an immune response toward a pre-selected antigen comprising the steps of:
i) removing a sample of antigen presenting cells from said mammal;
ii) exposing said antigen presenting cells to an effective CD40-expression-increasing amount of an agent;
iii) exposing said antigen presenting cells of step (ii) to a effective immune response inducing amount of a complex comprising a mammalian heat shock protein and said antigen; and
iv) returning said antigen presenting cells to said mammal.

26. The method of claim 25 wherein said mammalian heat shock protein is a hsp70 family member.

27. The method of claim 25 wherein said agent is an expression vector comprising a polynucleotide encoding CD40 or a heat shock protein binding fragment thereof.

28. The method of claim 25 wherein said agent is a calcium ionophore, a cytokine, or LPS.

29. The method of claim 28 wherein said cytokine is IL-1 alpha, TNF-alpha, IFN-gamma, IL-3 or GM-CSF.

30. The method of claim 25 wherein said complex is a covalent or non-covalent complex comprising said pre-selected antigen or an immunogenic fragment thereof and said mammalian heat shock protein or a CD40-binding fragment thereof.

31. The method of claim 30 wherein said non-covalent complex comprises a heat shock protein and a hybrid antigen, said hybrid antigen comprising a covalent conjugate between said pre-selected antigen or an immunogenic fragment thereof and a heat shock protein binding moiety.

32. The method of claim 31 wherein said heat shock protein binding moiety is a peptide or an organic molecule.

33. The method of claim 25 wherein said cell is a professional antigen presenting cell or a non-professional antigen presenting cell.

34. The method of claim 33 wherein said professional antigen presenting cell is a dendritic cell.

35. The method of claim 25 wherein said antigen presenting cells are isolated after step (ii) or step (iii).

36. The method of claim 25 wherein said antigen is an infectious disease antigen or a tumor antigen.

37. A method for enhancing the development in a mammal of tolerance to a pre-selected antigen comprising the steps of:
i) removing a sample of antigen presenting cells from said mammal:
ii) exposing said antigen presenting cells to an agent capable of increasing CD40 expression by said cell;
iii) exposing said antigen presenting cells of step (ii) to a effective immune response tolerizing amount of a complex comprising a mammalian heat shock protein and said antigen; and
iv) returning said antigen presenting cells to said mammal.

38. The method of claim 37 wherein said mammalian heat shock protein is a hsp70 family member.

39. The method of claim 37 wherein said agent is an expression vector comprising a polynucleotide encoding CD40 or a heat shock protein binding fragment thereof.

40. The method of claim 37 wherein said agent is a calcium ionophore, a cytokine, or LPS.

41. The method of claim 40 wherein said cytokine is IL-1 alpha, TNF-alpha, IFN-gamma, IL-3 or GM-CSF

42. The method of claim 37 wherein said heat shock protein is a covalent or noncovalent complex comprising said pre-selected antigen or an immunogenic fragment thereof and said heat shock protein.

43. The method of claim 42 wherein said non-covalent complex comprises a heat shock protein and a hybrid antigen, said hybrid antigen comprising a covalent conjugate between said pre-selected antigen or an immunogenic fragment thereof and a heat shock protein binding moiety.

44. The method of claim 43 wherein said heat shock protein binding moiety is a peptide or an organic molecule.

45. The method of claim 37 wherein said cell is a professional antigen presenting cell or a non-professional antigen presenting cell.

46. The method of claim 45 wherein said professional antigen presenting cell is a dendritic cell.

47. The method of claim 37 wherein said antigen presenting cells are isolated after step (ii) or step (iii).

48. The method of claim 37 wherein said antigen is an autoimmune antigen, a transplant antigen, or an allergen.

49. A method for enhancing the uptake of a complex of a mammalian heat shock protein and a pre-selected molecule by cells in vivo in an animal comprising administering to said animal an agent that increases CD40 expression by said cells.

50. The method of claim 49 wherein said agent is an expression vector comprising a polynucleotide encoding CD40 or a heat shock protein binding fragment thereof.

51. The method of claim 49 wherein said agent is a calcium ionophore, a cytokine, or LPS.

52. The method of claim 51 wherein said cytokine is IL-1 alpha, TNF-alpha, IFN-gamma, IL-3 or GM-CSF

53. The method of claim 49 wherein said complex is a covalent or noncovalent complex comprising said molecule and said heat shock protein.

54. The method of claim 53 wherein said non-covalent complex comprises a heat shock protein and a hybrid antigen, said hybrid antigen comprising a covalent conjugate between said molecule and a heat shock protein binding moiety.

55. The method of claim 54 wherein said heat shock protein binding moiety is a peptide or an organic molecule.

56. The method of claim 49 wherein said molecule is an infectious disease antigen or a tumor antigen.

57. A method for enhancing the development in a mammal of an immune response toward a pre-selected antigen comprising the steps of:
i) administering to said mammal an agent that increases CD40 expression by cells in said mammal; and
ii) administering to said mammal an effective immune response inducing amount of a complex comprising a mammalian heat shock protein and said pre-selected antigen.

58. The method of claim 57 wherein said agent is an expression vector comprising a polynucleotide encoding CD40 or a heat shock protein binding fragment thereof.

59. The method of claim 57 wherein said agent is a calcium ionophore, a cytokine, or LPS.

60. The method of claim 59 wherein said cytokine is IL-1 alpha, TNF-alpha, IFN-gamma, IL-3 or GM-CSF.

61. The method of claim 57 wherein said complex is a covalent or noncovalent complex comprising said pre-selected antigen or an immunogenic fragment thereof and said heat shock protein or a CD40-binding fragment thereof.

62. The method of claim 61 wherein said non-covalent complex comprises a heat shock protein and a hybrid antigen, said hybrid antigen comprising a covalent conjugate between said pre-selected antigen or an immunogenic fragment thereof and a heat shock protein binding moiety.

63. The method of claim 62 wherein said heat shock protein binding moiety is a peptide or an organic molecule.

64. The method of claim 57 wherein said antigen is an infectious disease antigen or a tumor antigen.

65. A method for decreasing the uptake of a complex of a mammalian heat shock protein and a molecule bound thereto by a cell expressing CD40 or a cell induced to express CD40 comprising exposing said cell to an agent that interferes with the binding of said mammalian heat shock protein to CD40 expressed by said cell.

66. The method of claim 65 wherein said agent comprises a CD40 binding partner.

67. The method of claim 66 wherein said CD40 binding partner is an antibody.

68. The method of claim 66 wherein said binding partner is CD40L of a CD40-binding fragment thereof.

69. The method of claim 66 wherein said binding partner is a CD40-binding fragment of a heat shock protein.

70. The method of claim 69 wherein said CD40-binding fragment of a heat shock protein comprises the fragment of human hsp70 starting from about amino acids 1-5 and extending to about amino acid 381, or any CD40-binding fragment thereof.

71. The method of claim 65 wherein said agent is a CD40 antisense oligonucleotide.

72. The method of claim 65 wherein said cell is a professional antigen presenting cell or a non-professional antigen presenting cell.

73. The method of claim 72 wherein said professional antigen presenting cell is a dendritic cell.

74. The method of claim 65 wherein said exposing is carried out in vitro, ex vivo, or in vivo.

75. A method for decreasing the development in a mammal of an immune response comprising the steps of:
i) removing a sample of antigen presenting cells from said mammal:
ii) exposing said antigen presenting cells to an agent that decreases CD40 expression by said cell; and
iii) returning said antigen presenting cells to said mammal.

76. The method of claim 75 wherein said agent is a CD40 antisense oligonucleotide.

77. A method for decreasing the uptake of a heat shock protein by cells in vivo in an animal comprising administering to said animal an agent that decreases CD40 expression by said cells.

78. The method of claim 77 wherein said agent is a CD40 antisense oligonucleotide.

79. A method for identifying a molecule capable of binding to the exoplasmic domain of CD40 comprising providing effective conditions for detectably measuring the extent of binding between a mammalian heat shock protein or a CD40-binding fragment thereof, and CD40 or a mammalian heat shock protein binding fragment thereof, exposing said molecule to said conditions, and correlating a change in said extent of said binding caused by said molecule with binding to the exoplasmic domain of CD40.

80. The method of claim 79 wherein said molecule does not have affinity for said heat shock protein or CD40-binding fragment thereof.

81. The method of claim 79 wherein said mammalian heat shock protein is a hsp70 family member.

82. The method of claim 79 wherein said CD40-binding fragment of a mammalian heat shock protein comprises the fragment of human hsp70 starting from about amino acids 1-5 and extending to about amino acid 381, or a fragment thereof.

83. The method of claim 79 wherein the mammalian heat shock protein binding fragment of CD40 is about amino acid 20 to about amino acid 212 of CD40.

84. The method of claim 79 wherein said molecule is a small organic molecule or a peptide.

85. A composition comprising a CD40-binding fragment of a mammalian heat shock protein and a pre-selected molecule.

86. The composition of claim 85 wherein said CD40-binding fragment does not comprise a peptide-binding domain of said mammalian heat shock protein.

87. The composition of claim 85 wherein said CD40-binding fragment is an N-domain of a mammalian heat shock protein.

88. The composition of claim 85 wherein said CD40-binding fragment is covalently bound to said pre-selected molecule.

89. The composition of claim 84 wherein said pre-selected molecule is an antigen or immunogenic fragment thereof.

90. The composition of claim 89 wherein said antigen is a cancer antigen, an infectious disease antigen, an autoimmune antigen, or a transplant antigen.

91. The composition of claim 85 wherein said CD40-binding fragment of a mammalian heat shock protein is amino acids 1-381 of human hsp70, or amino acids 5-381 of human hsp70, or a CD40-binding fragment thereof.

92. A composition comprising a molecule that binds to the exoplasmic domain of CD40, other than a heat shock protein or a fragment thereof, and a pre-selected molecule, said molecule that binds to the exoplasmic domain of CD40 identified by providing effective conditions for detectably measuring the extent of binding between a mammalian heat shock protein or a CD40-binding fragment thereof, and CD40 or a mammalian heat shock protein binding fragment thereof, exposing said molecule to said conditions, and correlating a change in said extent of said binding caused by said molecule with binding to the exoplasmic domain of CD40.

93. The method of claim 92 wherein said molecule does not have affinity for said heat shock protein or CD40-binding fragment thereof.

94. The composition of claim 92 wherein said molecule that binds to the exoplasmic domain of CD40 is covalently bound to said pre-selected molecule.

95. The composition of claim 92 wherein said pre-selected molecule is an antigen or immunogenic fragment thereof.

96. The composition of claim 95 wherein said antigen is a cancer antigen, an infectious disease antigen, an autoimmune antigen, or a transplant antigen.

97. A method for increasing the immune response to an immunogen in a mammalian patent comprising co-administering to said patient with said immunogen a CD40-binding fragment of a mammalian heat shock protein.

98. The method of claim 97 wherein said CD40-binding fragment of a mammalian heat shock protein comprises amino acids 1-381 of human hsp70, or amino acids 5-381 of human hsp70, or any CD40-binding fragment thereof.

99. A method for inducing an immune response in a mammal to a preselected antigen comprising exposing to phagocytes within, from, or intended to be administered to, said mammal a conjugate of a CD40-binding fragment of a heat shock protein and an antigen.

100. The method of claim 99 wherein said antigen is an infectious disease antigen, a tumor antigen, or an immunogenic portion or fragment thereof.

101. The method of claim 99 further comprising the step of increasing CD40 expression by phagocytes, said step carried out in vitro, ex vivo, or in vivo.

102. The method of claim 99 wherein said CD40-binding fragment of a mammalian heat shock protein comprises amino acids 1-381 of human hsp70, or amino acids 5-381 of human hsp70, or any CD40-binding fragment thereof.

103. A method for inducing an immune response in a mammal to a preselected antigen comprising exposing to phagocytes within, from, or intended to be administered to, said mammal a conjugate of a molecule other than a native heat shock protein or fragment thereof that binds to the exoplasmic domain of CD40 and said preselected antigen or an immunogenic portion thereof, said molecule that binds to the exoplasmic domain of CD40 identified by providing effective conditions for detectably measuring the extent of binding between a mammalian heat shock protein or a CD40-binding fragment thereof, and CD40 or a mammalian heat shock protein binding fragment thereof, exposing said molecule to said conditions, and correlating a change in said extent of said binding caused by said molecule with binding to the exoplasmic domain of CD40.

104. The method of claim 103 wherein said molecule does not have affinity for said heat shock protein or CD40-binding fragment thereof.

105. The method of claim 103 wherein said antigen is an infectious disease antigen or a tumor antigen.

106. The method of claim 103 further comprising the step of increasing CD40 expression by phagocytes, said step carried out in vitro, ex vivo, or in vivo.

107. A method for identifying a modified mammalian heat shock protein or CD40-binding fragment thereof with increased affinity for CD40 compared with native mammalian heat shock protein or a native CD40-binding fragment thereof, said method comprising providing effective conditions for detectably measuring the extent of binding between a native mammalian heat shock protein or a CD40-binding fragment thereof, and CD40 or a mammalian heat shock protein binding fragment thereof, exposing said modified heat shock protein or CD40-binding fragment thereof to said conditions, and correlating the extent of said binding by said modified mammalian heat shock protein or CD40-binding fragment thereof excess of that extent exhibited by said native mammalian heat shock protein of CD40-binding fragment thereof with increased affinity for binding to CD40.

108. Use of a CD40-binding fragment of a mammalian heat shock protein in a conjugate or complex with an antigen or immunogenic portion thereof for the preparation of a medicament for inducing an immune response against said antigen.

109. The use of claim 108 wherein said CD40-binding fragment of a mammalian heat shock protein comprises amino acids 1-381 of human hsp70, or amino acids 5-381 of human hsp70, or any CD40-binding fragment thereof.

110. The use of claim 108 wherein said pre-selected molecule is an antigen or an immunogenic fragment thereof.

111. The use of claim 108 wherein said antigen is a cancer antigen, an infectious disease antigen, an autoimmune antigen, or a transplant antigen.

112. The use of a CD40-binding molecule other than a native heat shock protein or fragment thereof in a conjugate or complex with an antigen or immunogenic portion thereof for the preparation of a medicament for inducing an immune response against said antigen.

113. The use of claim 112 wherein said CD40-binding molecule is identified by providing effective conditions for detectably measuring the extent of binding between a mammalian heat shock protein or a CD40-binding fragment thereof, and CD40 or a mammalian heat shock protein binding fragment thereof, exposing said molecule to said conditions, and correlating a change in said extent of said binding caused by said molecule with binding to the exoplasmic domain of CD40.

114. The use of claim 112 wherein said molecule does not have affinity for said heat shock protein or CD40-binding fragment thereof.
